(19) United States
(12) Patent Application Publication
(10) Pub. No.: US 2021/0307964 A1
(43) Pub. Date: Oct. 7, 2021

(54) METHOD, SYSTEM, AND APPARATUS FOR GENERATING THREE-DIMENSIONAL TREATMENT PATTERNS FOR LASER SURGERY OF GLAUCOMA

(71) Applicant: ViaLase, Inc., Aliso Viejo, CA (US)

(72) Inventors: Guy Holland, San Juan Capistrano, CA (US); Ferenc Raksi, Mission Viejo, CA (US); Hadi Srass, Yorba Linda, CA (US)

(21) Appl. No.: 16/838,858

(22) Filed: Apr. 2, 2020

(51) Int. Cl. A61F 9/008

(52) U.S. Cl. CPC A61F 9/00825; A61F 2009/00891; A61F 2009/00897; A61F 2009/00851; A61F 2009/00868

(57) ABSTRACT

A method of treating a target volume of ocular tissue of an irido-corneal angle of an eye with a laser source configured to deliver optical energy sufficient to affect ocular tissue at each of a plurality of instances during a scanning of a laser beam through a scanning pattern includes placing a focus of the laser beam at an initial depth in the target volume of ocular tissue; determining one or more instances at which to prevent a delivery of optical energy sufficient to affect ocular tissue; and delivering optical energy sufficient to affect ocular tissue at each of the plurality of instances during a scanning of the laser beam through the scanning pattern except for the determined one or more instances, to thereby affect an initial treatment plane of the target volume of ocular tissue at the initial depth.

### TECHNICAL FIELD

The present disclosure relates generally to the field of medical devices and treatment of diseases in ophthalmology, and more particularly to systems, apparatuses, and methods for generating three-dimensional treatment patterns for tissues, especially ocular tissue structures in the irido-corneal angle of the eye, for laser surgery treatment of glaucoma.

### BACKGROUND

Before describing the different types of glaucoma and current diagnosis and treatments options, a brief overview of the anatomy of the eye is provided.

Anatomy of the Eye

With reference to FIGS. 1-3, the outer tissue layer of the eye 1 includes a sclera 2 that provides the structure of the eye's shape. In front of the sclera 2 is a cornea 3 that is comprised of transparent layers of tissue that allow light to enter the interior of the eye. Inside the eye 1 is a crystalline lens 4 that is connected to the eye by fiber zonules 5, which are connected to the ciliary body 6. Between the crystalline lens 4 and the cornea 3 is an anterior chamber 7 that contains a flowing clear liquid called aqueous humor 8. Encircling the perimeter of the crystalline lens 4 is an iris 9 which forms a pupil around the approximate center of the crystalline lens. A posterior chamber 23 is an annular volume behind the iris 9 and bounded by the ciliary body 6, fiber zonules 5, and the crystalline lens 4. The vitreous humor 10 is located between the crystalline lens 4 and the retina 11. Light entering the eye is optically focused through the cornea 3 and crystalline lens.

With reference to FIG. 2, the corneoscleral junction of the eye is the portion of the anterior chamber 7 at the intersection of the iris 9, the sclera 2, and the cornea 3. The anatomy of the eye 1 at the corneoscleral junction includes a trabecular meshwork 12. The trabecular meshwork 12 is a fibrous network of tissue that encircles the iris 9 within the eye 1. In simplified, general terms the tissues of the corneoscleral junction are arranged as follows: the iris 9 meets the ciliary body 6, the ciliary body meets with the underside of the scleral spur 14, the top of the scleral spur serves as an attachment point for the bottom of the trabecular meshwork 12. The ciliary body is present mainly in the posterior chamber, but also extends into the very corner of the anterior chamber 7. The network of tissue layers that make up the trabecular meshwork 12 are porous and thus present a pathway for the egress of aqueous humor 8 flowing from the anterior chamber 7. This pathway may be referred to herein as an aqueous humor outflow pathway, an aqueous outflow pathway, or simply an outflow pathway.

Referring to FIG. 3, the pathway formed by the pores in the trabecular meshwork 12 connect to a set of thin porous tissue layers called the uveal 15, the corneoscleral meshwork 16, and the juxtacanalicular tissue 17. The juxtacanalicular tissue 17, in turn, abuts a structure called Schlemm's canal 18. The Schlemm's canal 18 carries a mixture of aqueous humor 8 and blood from the surrounding tissue to drain into the venous system though a system of collector channels 19. As shown in FIG. 2, the vascular layer of the eye, referred to as the choroid 20, is next to the sclera 2. A space, called the suprachoroidal space 21, may be present between the choroid 20 and the sclera 2. The general region near the periphery of the wedge between the cornea 3 and the iris 9, running circumferentially is called the irido-corneal angle 13. The irido-corneal angle 13 may also be referred to as the corneal angle of the eye or simply the angle of the eye. The ocular tissues illustrated in FIG. 3 are all considered to be within the irido-corneal angle 13.

With reference to FIG. 4, two possible outflow pathways for the movement of aqueous humor 8 include a trabecular outflow pathway 40 and a uveoscleral outflow pathway 42. Aqueous humor 8, which is produced by the ciliary body 6, flows from the posterior chamber 23 through the pupil into the anterior chamber 7, and then exits the eye through one or more of the two different outflow pathways 40, 42. Approximately 90% of the aqueous humor 8 leaves via the trabecular outflow pathway 40 by passing through the trabecular meshwork 12, into the Schlemm's canal 18 and through one or more plexus of collector channels 19 before draining through a drain path 41 into the venous system. Any remaining aqueous humor 8 leaves primarily through the uveoscleral outflow pathway 42. The uveoscleral outflow pathway 42 passes through the ciliary body 6 face and iris root into the suprachoroidal space 21 (shown in FIG. 2). Aqueous humor 8 drains from the suprachoroidal space 21, from which it can be drained through the sclera 2.

The intra-ocular pressure of the eye depends on the aqueous humor 8 outflow through the trabecular outflow pathway 40 and the resistance to outflow of aqueous humor through the trabecular outflow pathway. The intra-ocular pressure of the eye is largely independent of the aqueous humor 8 outflow through the uveoscleral outflow pathway 42. Resistance to the outflow of aqueous humor 8 through the trabecular outflow pathway 40 may lead to elevated intra-ocular pressure of the eye, which is a widely recognized risk factor for glaucoma. Resistance through the trabecular outflow pathway 40 may increase due a collapsed or malfunctioning Schlemm's canal 18 and trabecular meshwork 12.

Referring to FIG. 5, as an optical system, the eye 1 is represented by an optical model described by idealized centered and rotationally symmetrical surfaces, entrance and exit pupils, and six cardinal points: object and image space focal points, first and second principal planes, and first and second nodal points. Angular directions relative to the human eye are often defined with respect to an optical axis 24, a visual axis 26, a pupillary axis 28 and a line of sight 29 of the eye. The optical axis 24 is the symmetry axis, the line connecting the vertices of the idealized surfaces of the eye. The visual axis 26 connects the foveal center 22 with the first and second nodal points to the object. The line of sight 29 connects the fovea through the exit and entrance pupils to the object. The pupillary axis 28 is normal to the anterior surface of the cornea 3 and directed to the center of the entrance pupil. These axes of the eye differ from one another only by a few degrees and fall within a range of what is generally referred to as the direction of view.

Glaucoma

Glaucoma is a group of diseases that can harm the optic nerve and cause vision loss or blindness. It is the leading cause of irreversible blindness. Approximately 80 million people are estimated to have glaucoma worldwide and of these, approximately 6.7 million are bilaterally blind. More than 2.7 million Americans over age 40 have glaucoma. Symptoms start with loss of peripheral vision and can progress to blindness.

There are two forms of glaucoma, one is referred to as closed-angle glaucoma, the other as open-angled glaucoma. With reference to FIGS. 1-4, in closed-angle glaucoma, the iris 9 in a collapsed anterior chamber 7 may obstruct and close off the flow of aqueous humor 8. In open-angle glaucoma, which is the more common form of glaucoma, the permeability of ocular tissue may be affected by irregularities in the juxtacanalicular tissue 17 and inner wall of Schlemm's canal 18a, blockage of tissue in the irido-corneal angle 13 along the trabecular outflow pathway 40.

As previously stated, elevated intra-ocular pressure (TOP) of the eye, which damages the optic nerve, is a widely recognized risk factor for glaucoma. However, not every person with increased eye pressure will develop glaucoma, and glaucoma can develop without increased eye pressure. Nonetheless, it is desirable to reduce elevated TOP of the eye to reduce the risk of glaucoma.

Methods of diagnosing conditions of the eye of a patient with glaucoma include visual acuity tests and visual field tests, dilated eye exams, tonometry, i.e. measuring the intra-ocular pressure of the eye, and pachymetry, i.e. measuring the thickness of the cornea. Deterioration of vision starts with the narrowing of the visual field and progresses to total blindness. Imaging methods include slit lamp examination, observation of the irido-corneal angle with a gonioscopic lens and optical coherence tomography (OCT) imaging of the anterior chamber and the retina

Once diagnosed, some clinically proven treatments are available to control or lower the intra-ocular pressure of the eye to slow or stop the progress of glaucoma. The most common treatments include: 1) medications, such as eye drops or pills, 2) laser surgery, and 3) traditional surgery. Treatment usually begins with medication. However, the efficacy of medication is often hindered by patient non-compliance. When medication does not work for a patient, laser surgery is typically the next treatment to be tried. Traditional surgery is invasive, more high risk than medication and laser surgery, and has a limited time window of effectiveness. Traditional surgery is thus usually reserved as a last option for patients whose eye pressure cannot be controlled with medication or laser surgery.

Laser Surgery

With reference to FIG. 2, laser surgery for glaucoma targets the trabecular meshwork 12 to decrease aqueous humor 8 flow resistance. Common laser treatments include Argon Laser Trabeculoplasty (ALT), Selective Laser Trabeculoplasty (SLT) and Excimer Laser Trabeculostomy (ELT).

ALT was the first laser trabeculoplasty procedure. During the procedure, an argon laser of 514 nm wavelength is applied to the trabecular meshwork 12 around 180 degrees of the circumference of the irido-corneal angle 13. The argon laser induces a thermal interaction with the ocular tissue that produces openings in the trabecular meshwork 12. ALT, however, causes scarring of the ocular tissue, followed by inflammatory responses and tissue healing that may ultimately close the opening through the trabecular meshwork 12 formed by the ALT treatment, thus reducing the efficacy of the treatment. Furthermore, because of this scarring, ALT therapy is typically not repeatable.

SLT is designed to lower the scarring effect by selectively targeting pigments in the trabecular meshwork 12 and reducing the amount of heat delivered to surrounding ocular tissue. During the procedure, a solid-state laser of 532 nm wavelength is applied to the trabecular meshwork 12 between 180 to 360 degrees around the circumference of the irido-corneal angle 13 to remove the pigmented cells lining the trabeculae which comprise the trabecular meshwork. The collagen ultrastructure of the trabecular meshwork is preserved during SLT. 12. SLT treatment can be repeated, but subsequent treatments have lower effects on TOP reduction.

ELT uses a 308 nm wavelength ultraviolet (UV) excimer laser and non-thermal interaction with ocular tissue to treat the trabecular meshwork 12 and inner wall of Schlemm's canal in a manner that does not invoke a healing response. Therefore, the TOP lowering effect lasts longer. However, because the UV light of the laser cannot penetrate deep into the eye, the laser light is delivered to the trabecular meshwork 12 via an optical fiber inserted into the eye 1 through an opening and the fiber is brought into contact with the trabecular meshwork. The procedure is highly invasive and is generally practiced simultaneously with cataract procedures when the eye is already surgically open. Like ALT and SLT, ELT also lacks control over the amount of TOP reduction.

None of these existing laser treatments represents an ideal treatment for glaucoma. Accordingly, what is needed are systems, apparatuses, and method for laser surgery treatment of glaucoma that effectively reduce TOP non-invasively without significant scarring of tissue, so the treatment may be completed in a single procedure and repeated at a later time if necessary.

Such systems, apparatuses, and methods for laser surgery treatment of glaucoma are disclosed in U.S. patent application Ser. No. 16/036,833, filed Jul. 16, 2018, for “Integrated Surgical System and Method for Treatment in the Irido-Corneal Angle of the Eye,” and U.S. patent application Ser. No. 16/125,588, filed Sep. 7, 2018, for “Non-Invasive and Minimally Invasive Laser Surgery for the Reduction of Intraocular Pressure in the Eye,” the entire disclosures of which are incorporated herein by reference. These applications disclose laser surgery techniques in which volumes of ocular tissue are treated by scanning a laser through a three-dimensional (3D) pattern while delivering optical energy sufficient to affect ocular tissue. 3D scan patterns, however, are computationally complex and can take a long time to develop. Accordingly, what is further needed in the field of laser treatment of glaucoma are systems, apparatuses, and methods that enable efficient development of 3D scanning patterns.

### SUMMARY

The present disclosure relates to a method of treating a target volume of ocular tissue of an irido-corneal angle of an eye with a laser source configured to deliver optical energy sufficient to affect ocular tissue at each of a plurality of instances during a scanning of a laser beam through a scanning pattern. The scanning pattern may be a raster pattern, a circular pattern, a spiral pattern, or a non-symmetric pattern. The method includes placing a focus of the laser beam at an initial depth in the target volume of ocular tissue, and determining one or more instances at which to prevent a delivery of optical energy sufficient to affect ocular tissue. The method further includes delivering optical energy sufficient to affect ocular tissue at each of the plurality of instances during a scanning of the laser beam through the scanning pattern—except for the determined one or more instances. Scanning and delivering optical energy in this manner affects an initial treatment plane of the target volume of ocular tissue at the initial depth. The instances where ocular tissue is affected and the determined instances where ocular tissue is not affected are pre-computed by a processor and define a treatment pattern.

The method may further include placing the focus of the laser beam at a subsequent depth in the target volume of ocular tissue; determining one or more instances at which to prevent a delivery of optical energy sufficient to affect ocular tissue; and delivering optical energy sufficient to affect ocular tissue at each of the plurality of instances during a scanning of the laser beam through the pattern—except for the determined one or more instances. Scanning and delivering optical energy in this manner affects a subsequent treatment plane of the target volume of ocular tissue at the subsequent depth. The initial treatment plane and the at least one subsequent treatment plane are adjacent each other, and the combination of these adjacent planes defines the target volume of ocular tissue.

The combination of the initial treatment plane and the at least one subsequent treatment plane may form a three-dimensional (3D) model or treatment pattern comprising a plurality of two-dimensional (2D) layers or treatment planes. A 3D model and its corresponding plurality of 2D layers may take any shape. For example, a 3D model may be cylinder formed by a plurality of circular 2D layers of the same diameter. In another example, a 3D model may be a cone formed by a plurality of circular 2D layers of the different diameters, where the diameter is reduced in size at leach successive layer along the length of the cone.

The geometries of the 2D layers and corresponding 3D models are defined by image filters that identify the determined one or more of the instances during the scanning of a laser beam at which to prevent a delivery of optical energy sufficient to affect ocular tissue. Image filter information may be embodied in one of a 3D CAD software file, a stereo lithography file, an image file, a plurality of image files, an spreadsheet file, and an electronic tabulation of spatial coordinates.

The information in an image filter maps to the instances of a scanning pattern and the delivery of optical energy is controlled based on the mapping. For example, the information may identify at which instances the laser pulses should be blocked at the output end of the laser source. The determined instances while scanning the laser beam through the scanning pattern at the initial depth, and the determined instances while scanning the laser beam through the pattern at the subsequent depth may be identical. In these cases, each 2D layer has the same shape and size and the 3D model is uniform along its length, like a cylinder or rectangular bar. The determined instances while scanning the laser beam through the scanning pattern at the initial depth, and the determined instances while scanning the laser beam through the pattern at the subsequent depth may not be identical. In these cases, each 2D layer or sets of 2D layers may be the same shape but a different size, and the 3D model is nonuniform along its length, like a cone or an hourglass or a funnel.

The present disclosure also relates to an integrated surgical system for treating a target volume of ocular tissue of an irido-corneal angle of an eye. The system includes a first optical subsystem, a second optical system, and a control system. The first optical subsystem includes a focusing objective configured to be coupled to a cornea of the eye. The second optical subsystem includes a laser source configured to output a laser beam and to deliver optical energy sufficient to affect ocular tissue at each of a plurality of instances during a scanning of the laser beam through a scanning pattern, and a plurality of components configured to one or more of condition, scan, and direct the laser beam. The control system is coupled to one or more of the first optical subsystem and the second optical subsystem and is configured to control the focus, scan and optical energy delivery of the laser source to place a focus of the beam laser at an initial depth in the target volume of ocular tissue, and determine one or more instances at which to prevent a delivery of optical energy sufficient to affect ocular tissue. The control system is further configured to control the focus, scan and optical energy delivery of the laser source to deliver optical energy sufficient to affect ocular tissue at each of the plurality of instances during a scanning of the laser beam through the scanning pattern—except for the determined one or more instances, to thereby affect an initial treatment plane of the target volume of ocular tissue at the initial depth. The instances where ocular tissue is affected and the determined instances where ocular tissue is not affected are pre-computed by a processor and define a treatment pattern that is used by the control system to control the focus, scan and optical energy delivery of the laser source.

The control system may be further configured to place the focus of the laser beam at a subsequent depth in the target volume of ocular tissue; determine one or more instances at which to prevent a delivery of optical energy sufficient to affect ocular tissue; and deliver optical energy sufficient to affect ocular tissue at each of the plurality of instances during a scanning of the laser beam through the pattern—except for the determined one or more instances. Scanning and delivering optical energy in this manner affects a subsequent treatment plane of the target volume of ocular tissue at the subsequent depth. The initial treatment plane and the at least one subsequent treatment plane are adjacent each other, and the combination of these adjacent planes defines the target volume of ocular tissue.

The present disclosure also relates to an apparatus for controlling delivery of optical energy during a scanning of a laser beam through a three-dimensional (3D) scanning pattern. The apparatus includes a memory and a controller coupled to the memory. The controller is configured to obtain information corresponding to a pre-computed 3D treatment pattern that identifies first instances of the 3D scanning pattern at which a delivery of optical energy sufficient to affect ocular tissue is to be allowed, and second instances of the 3D scanning pattern at which a delivery of optical energy sufficient to affect ocular tissue is to be prevented. The controller is further configured to deliver optical energy sufficient to affect ocular tissue at each of the first instances during a scanning of the laser beam through the 3D scanning pattern, and to prevent a delivery of optical energy sufficient to affect ocular tissue at each of the second instances during a scanning of the laser beam through the 3D scanning pattern. The information obtained by the controller comprises 3D coordinate locations of the first instances and the second instances within the 3D scanning pattern, and a binary bit of a first value for each of the first instances, and a binary bit of a second value for each of the second instances.

It is understood that other aspects of methods and systems will become apparent to those skilled in the art from the following detailed description, wherein various aspects of apparatuses and methods are shown and described by way of illustration. As will be realized, these aspects may be implemented in other and different forms and its several details are capable of modification in various other respects. Accordingly, the drawings and detailed description are to be regarded as illustrative in nature and not as restrictive.

### BRIEF DESCRIPTION OF THE DRAWINGS

Various aspects of systems, apparatuses, and methods will now be presented in the detailed description by way of example, and not by way of limitation, with reference to the accompanying drawings, wherein:

FIG. 1 is a sectional schematic illustration of a human eye and its interior anatomical structures.

FIG. 2 is a sectional schematic illustration of the irido-corneal angle of the eye of FIG. 1.

FIG. 3 is a sectional schematic illustration detailing anatomical structures in the irido-corneal angle of FIG. 2, including the trabecular meshwork, Schlemm's canal, and one or more collector channels branching from the Schlemm's canal.

FIG. 4 is a sectional schematic illustration of various outflow pathways for aqueous humor through the trabecular meshwork, Schlemm's canal, and collector channels of FIG. 3.

FIG. 5 is a sectional schematic illustration of a human eye showing various axes associated with the eye.

FIG. 6 is a sectional schematic illustration of an angled beam path along which one or more light beams may access the irido-corneal angle of the eye.

FIG. 7 is a block diagram of an integrated surgical system for non-invasive glaucoma surgery including a control system, a femtosecond laser source, an OCT imaging apparatus, a microscope, beam conditioners and scanners, beam combiners, a focusing objective, and a patient interface.

FIG. 8 is a detailed block diagram of the integrated surgical system of FIG. 7.

FIGS. 9a and 9b are schematic illustrations of the focusing objective of the integrated surgical system of FIG. 7 coupled to (FIG. 9a) and decoupled from (FIG. 9b) the patient interface of the integrated surgical system of FIG. 7.

FIG. 9c is a schematic illustration of components of the focusing objective and the patient interface included in FIGS. 9a and 9b.

FIGS. 10a and 10b are schematic illustrations of components of the integrated surgical system of FIGS. 7 and 8 functionally arranged to form a first optical system and a second optical subsystem that enable access to the to the irido-corneal angle along the angled beam path of FIG. 6.

FIG. 10c is a schematic illustration of a beam passing through the first optical subsystem of FIGS. 10a and 10b and into the eye.

FIGS. 11a through 11h are illustrations of example laser treatment patterns formed by a number of treatment planes and used by the integrated surgical system of FIG. 7 to treat a target volume of tissue.

FIG. 12 is a schematic illustration of an example scanning pattern used by the integrated surgical system of FIG. 7 when scanning a laser focus and comprised of a two-dimensional array of instances during which optical energy may be delivered to tissue.

FIG. 13 is a three-dimensional schematic illustration of anatomical structures in the irido-corneal angle, including the trabecular meshwork, Schlemm's canal, a collector channel branching from the Schlemm's canal, and a cubic surgical volume of ocular tissue to be treated by the integrated surgical system of FIG. 7.

FIG. 14 is a two-dimensional schematic illustration of anatomical structures in the irido-corneal angle and a three-dimensional illustration of a laser treatment pattern to be applied by the integrated surgical system of FIG. 7 to affect the surgical volume of ocular tissue shown in FIG. 13.

FIG. 15 is an illustration of a horizontal scan that includes a delivery of optical energy at each instance the pattern of FIG. 12.

FIG. 16 is a three-dimensional schematic illustration of FIG. 11 subsequent to treatment of the surgical volume of ocular tissue by a laser based on the laser treatment pattern of FIG. 14 that forms an opening between the Schlemm's canal and the anterior chamber.

FIG. 17 is a three-dimensional schematic illustration of anatomical structures in the irido-corneal angle, including the trabecular meshwork, Schlemm's canal, a collector channel branching from the Schlemm's canal, and a cylindrical surgical volume of ocular tissue to be treated by the integrated surgical system of FIG. 7.

FIG. 18 is a two-dimensional schematic illustration of anatomical structures in the irido-corneal angle and a three-dimensional illustration of a laser treatment pattern to be applied by the integrated surgical system of FIG. 7 to affect the surgical volume of ocular tissue shown in FIG. 17.

FIG. 19 is an illustration of a horizontal scan that includes preventing a delivery of optical energy at certain instances, while allowing for the delivery of optical energy at other instances in the pattern of FIG. 12.

FIG. 20 is a three-dimensional schematic illustration of FIG. 11 subsequent to treatment of the surgical volume of ocular tissue by a laser based on the laser treatment pattern of FIG. 18 that forms an opening between the Schlemm's canal and the anterior chamber.

FIG. 21 is a flow chart of a method of treating a target volume of ocular tissue of an irido-corneal angle of an eye with a laser source configured to deliver optical energy sufficient to affect ocular tissue at a plurality of instances during a scanning of a laser beam through a scanning pattern.

FIGS. 22a and 22b are illustrations of example treatment patterns comprising a number of individual geometric structures.

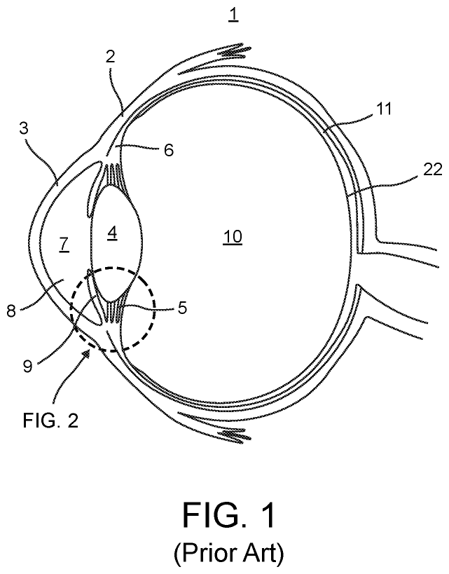

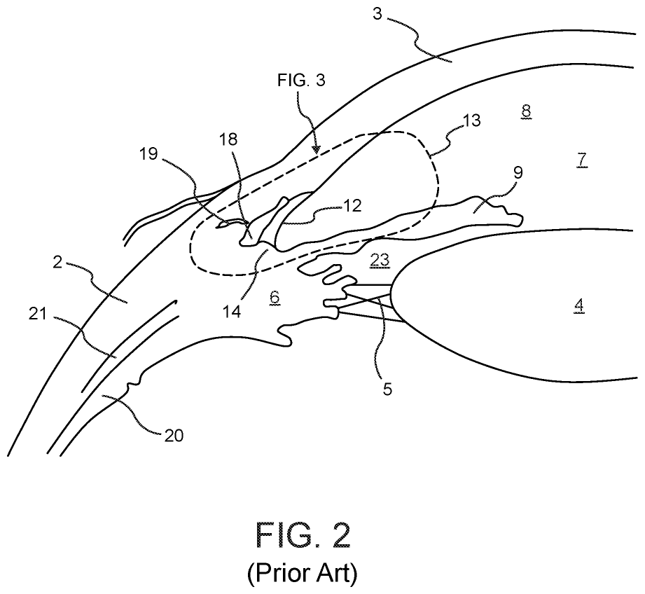

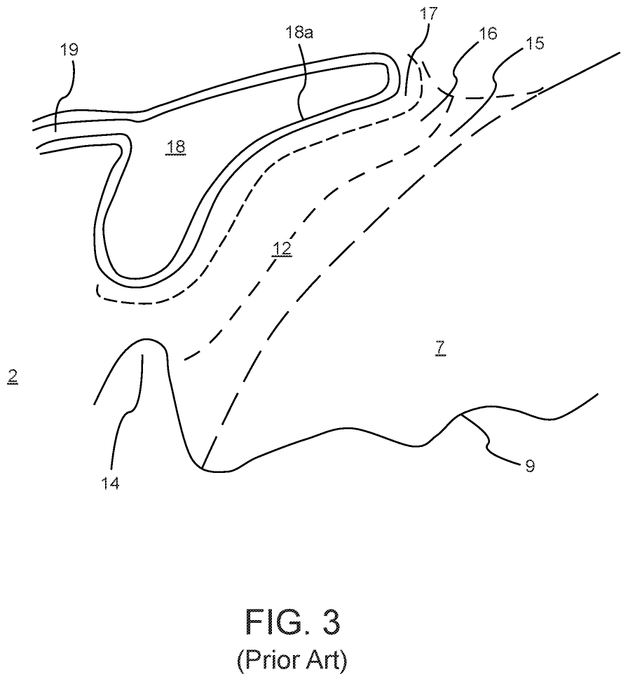

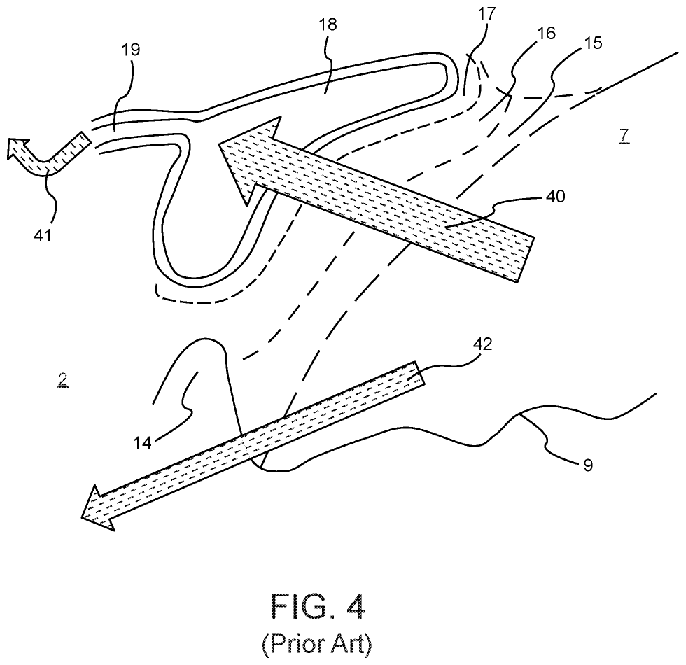

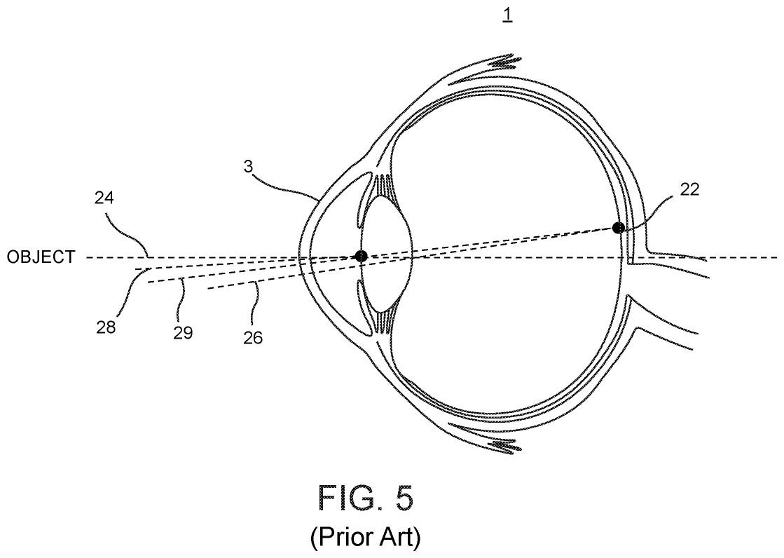

### DETAILED DESCRIPTION

Disclosed herein are systems and methods for safely and effectively reducing intra-ocular pressure (TOP) in the eye to either treat or reduce the risk of glaucoma. The systems and methods enable access to the irido-corneal angle of the eye and use laser surgery techniques to treat abnormal ocular tissue conditions within the irido-corneal angle that may be causing elevated IOP. The system and method also enable the locating of a focus of a femtosecond laser at or near a target structure of ocular tissue for laser surgery.

An integrated surgical system disclosed herein is configured to reduce intraocular pressure in an eye having a cornea, an anterior chamber, and an irido-corneal angle comprising an aqueous humor outflow pathway formed of a trabecular meshwork, a Schlemm's canal, and one or more collector channels branching from the Schlemm's canal. The integrated surgical system includes a first optical subsystem and a second optical subsystem. The first optical subsystem includes a window configured to be coupled to the cornea and an exit lens configured to be coupled to the window. The second optical subsystem includes an optical coherence tomography (OCT) imaging apparatus configured to output an OCT beam, a laser source configured to output a laser beam, and a plurality of components, e.g., lenses and mirrors, configured to condition, combine, or direct the OCT beam and the laser beam toward the first optical subsystem.

The integrated surgical system also includes a control system coupled to the OCT imaging apparatus, the laser source, and the second optical subsystem. The control is configured to instruct the OCT imaging apparatus to output an OCT beam and the laser source to output a laser beam, for delivery through the cornea, and the anterior chamber into the irido-corneal angle. The control system of the integrated surgical system is further configured to instruct the laser source to modify a volume of ocular tissue within the outflow pathway to reduce a pathway resistance present in one or more of the trabecular meshwork, the Schlemm's canal, and the one or more collector channels by applying the laser beam to ocular tissue defining the volume to thereby cause photo-disruptive interaction with the ocular tissue to reduce the pathway resistance or create a new outflow pathway.

The laser source may be a femtosecond laser. Femtosecond lasers provide non-thermal photo-disruption interaction with ocular tissue to avoid thermal damage to surrounding tissue. Further, unlike other surgical methods, with femtosecond laser treatment opening surface incisions penetrating the eye can be avoided, enabling a non-invasive treatment. Instead of performing the treatment in a sterile surgical room, the non-invasive treatment can be performed in a non-sterile outpatient facility.

An additional imaging component may be included the integrated surgical system to provide direct visual observation of the irido-corneal angle along an angle of visual observation. For example, a microscope or imaging camera may be included to assist the surgeon in the process of docking the eye to the patient interface or an immobilizing device, location of ocular tissues in the eye and observing the progress of the surgery. The angle of visual observation can also be along the angled beam path 30 to the irido-corneal angle 13 through the cornea 3 and the anterior chamber 7.

OCT Imaging

OCT imaging may be used to determine the structural and geometrical conditions of the anterior chamber 7, to assess possible obstruction of the trabecular outflow pathway 40 and to determine the accessibility of the ocular tissue for treatment. As previously described, the iris 9 in a collapsed anterior chamber 7 may obstruct and close off the flow of aqueous humor 8, resulting in closed-angle glaucoma. In open-angle glaucoma, where the macroscopic geometry of the angle is normal, the permeability of ocular tissue may be affected, by blockage of tissue along the trabecular outflow pathway 40 or by the collapse of the Schlemm's canal 18 or collector channels 19.

OCT imaging can provide the necessary spatial resolution, tissue penetration and contrast to resolve microscopic details of ocular tissue. When scanned, OCT imaging can provide two-dimensional (2D) cross-sectional images of the ocular tissue. As another aspect of the integrated surgical system, 2D cross-sectional images may be processed and analyzed to determine the size, shape and location of structures in the eye for surgical targeting. It is also possible to reconstruct three-dimensional (3D) images from a multitude of 2D cross-sectional images but often it is not necessary. Acquiring, analyzing and displaying 2D images is faster and can still provide all information necessary for precise surgical targeting.

Femtosecond Laser Source

The preferred surgical component of the integrated surgical system disclosed herein is a femtosecond laser. A femtosecond laser provides highly localized, non-thermal photo-disruptive laser-tissue interaction with minimal collateral damage to surrounding ocular tissue. Photo-disruptive interaction of the laser is utilized in optically transparent tissue. The principal mechanism of laser energy deposition into the ocular tissue is not by absorption but by a highly nonlinear multiphoton process. This process is effective only at the focus of the pulsed laser where the peak intensity is high. Regions where the beam is traversed but not at the focus are not affected by the laser. Therefore, the interaction region with the ocular tissue is highly localized both transversally and axially along the laser beam. The process can also be used in weakly absorbing or weakly scattering tissue. While femtosecond lasers with photo-disruptive interactions have been successfully used in ophthalmic surgical systems and commercialized in other ophthalmic laser procedures, none have been used in an integrated surgical system that accesses the irido-corneal angle.

In known refractive procedures, femtosecond lasers are used to create corneal flaps, pockets, tunnels, arcuate incisions, lenticule shaped incisions, partial or fully penetrating corneal incisions for keratoplasty. For cataract procedures the laser creates a circular cut on the capsular bag of the eye for capsulotomy and incisions of various patterns in the lens for braking up the interior of the crystalline lens to smaller fragments to facilitate extraction. Entry incisions through the cornea opens the eye for access with manual surgical devices and for insertions of phacoemulsification devices and intra-ocular lens insertion devices. Several companies have commercialized such surgical systems, among them the IntraLase system now available from Johnson & Johnson Vision, Santa Ana, Calif., The LenSx and WaveLight systems from Alcon, Fort Worth, Tex., other surgical systems from Bausch and Lomb, Rochester, N.Y., Carl Zeiss Meditec AG, Germany, Ziemer, Port, Switzerland, and LENSAR, Orlando, Fla.

These existing systems are developed for their specific applications, for surgery in the cornea, and the crystalline lens and its capsular bag and are not capable of performing surgery in the irido-corneal angle 13 for several reasons. First, the irido-corneal angle 13 is not accessible with these surgical laser systems because the irido-corneal angle is too far out in the periphery and is outside of surgical range of these systems. Second, the angle of the laser beam from these systems, which is along the optical axis to the eye 24, is not appropriate to reaching the irido-corneal angle 13, where there is significant scattering and optical distortion at the applied wavelength. Third, any imaging capabilities these systems may have do not have the accessibility, penetration depth and resolution to image the tissue along the trabecular outflow pathway 40 with sufficient detail and contrast.

Clear access to the irido-corneal angle 13 is provided along the angled beam path 30. The tissue, e.g., cornea 3 and the aqueous humor 8 in the anterior chamber 7, along this angled beam path 30 is transparent for wavelengths from approximately 400 nm to 2500 nm and femtosecond lasers operating in this region can be used. Such mode locked lasers work at their fundamental wavelength with Titanium, Neodymium or Ytterbium active material. Non-linear frequency conversion techniques known in the art, frequency doubling, tripling, sum and difference frequency mixing techniques, optical parametric conversion can convert the fundamental wavelength of these lasers to practically any wavelength in the above mentioned transparent wavelength range of the cornea.

Existing ophthalmic surgical systems apply lasers with pulse durations longer than 1 ns have higher photo-disruption threshold energy, require higher pulse energy and the dimension of the photo-disruptive interaction region is larger, resulting in loss of precision of the surgical treatment. When treating the irido-corneal angle 13, however, higher surgical precision is required. To this end, the integrated surgical system may be configured to apply lasers with pulse durations from 10 femtosecond (fs) to 1 nanosecond (ns) for generating photo-disruptive interaction of the laser beam with ocular tissue in the irido-corneal angle 13. While lasers with pulse durations shorter than 10 fs are available, such laser sources are more complex and more expensive. Lasers with the described desirable characteristics, e.g., pulse durations from 10 femtosecond (fs) to 1 nanosecond (ns), are commercially available from multiple vendors, such as Newport, Irvine, Calif., Coherent, Santa Clara, Calif., Amplitude Systems, Pessac, France, NKT Photonics, Birkerod, Denmark, and other vendors.

Accessing the Irido-corneal Angle

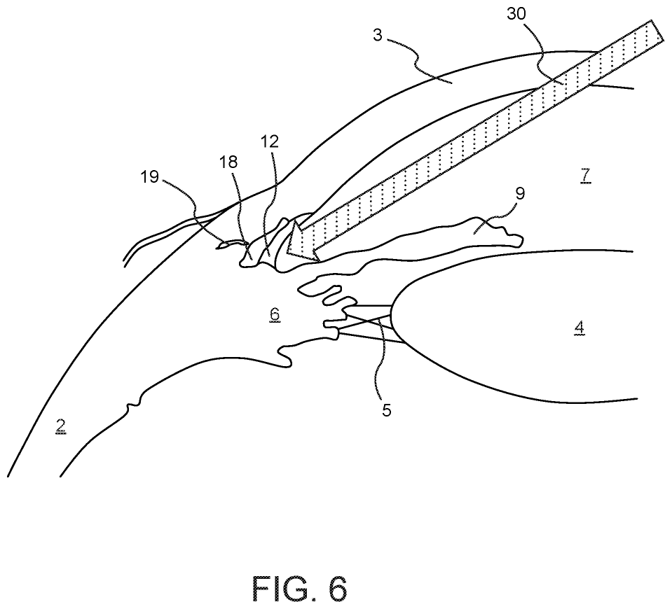

An important feature afforded by the integrated surgical system is access to the targeted ocular tissue in the irido-corneal angle 13. With reference to FIG. 6, the irido-corneal angle 13 of the eye may be accessed via the integrated surgical system along an angled beam path 30 passing through the cornea 3 and through the aqueous humor 8 in the anterior chamber 7. For example, one or more of an imaging beam, e.g., an OCT beam and/or a visual observation beam, and a laser beam may access the irido-corneal angle 13 of the eye along the angled beam path 30.

An optical system disclosed herein is configured to direct a light beam to an irido-corneal angle 13 of an eye along an angled beam path 30. The optical system includes a first optical subsystem and a second optical subsystem. The first optical subsystem includes a window formed of a material with a refractive index n_{w} and has opposed concave and convex surfaces. The first optical subsystem also includes an exit lens formed of a material having a refractive index nₓ. The exit lens also has opposed concave and convex surfaces. The concave surface of the exit lens is configured to couple to the convex surface of the window to define a first optical axis extending through the window and the exit lens. The concave surface of the window is configured to detachably couple to a cornea of the eye with a refractive index n_{c} such that, when coupled to the eye, the first optical axis is generally aligned with the direction of view of the eye.

The second optical subsystem is configured to output a light beam, e.g., an OCT beam or a laser beam. The optical system is configured so that the light beam is directed to be incident at the convex surface of the exit lens along a second optical axis at an angle α that is offset from the first optical axis. The respective geometries and respective refractive indices nₓ, and n_{w} of the exit lens and window are configured to compensate for refraction and distortion of the light beam by bending the light beam so that it is directed through the cornea 3 of the eye toward the irido-corneal angle 13. More specifically, the first optical system bends the light beam to that the light beam exits the first optical subsystem and enters the cornea 3 at an appropriate angle so that the light beam progresses through the cornea and the aqueous humor 8 in a direction along the angled beam path 30 toward the irido-corneal angle 13.

Accessing the irido-corneal angle 13 along the angled beam path 30 provides several advantages. An advantage of this angled beam path 30 to the irido-corneal angle 13 is that the OCT beam and laser beam passes through mostly clear tissue, e.g., the cornea 3 and the aqueous humor 8 in the anterior chamber 7. Thus, scattering of these beams by tissue is not significant. With respect to OCT imaging, this enables the use of shorter wavelength, less than approximately 1 micrometer, for the OCT to achieve higher spatial resolution. An additional advantage of the angled beam path 30 to the irido-corneal angle 13 through the cornea 3 and the anterior chamber 7 is the avoidance of direct laser beam or OCT beam light illuminating the retina 11. As a result, higher average power laser light and OCT light can be used for imaging and surgery, resulting in faster procedures and less tissue movement during the procedure.

Another important feature provided by the integrated surgical system is access to the targeted ocular tissue in the irido-corneal angle 13 in a way that reduces beam discontinuity. To this end, the window and exit lens components of the first optical subsystem are configured to reduce the discontinuity of the optical refractive index between the cornea 3 and the neighboring material and facilitate entering light through the cornea at a steep angle.

Having thus generally described the integrated surgical system and some of its features and advantages, a more detailed description of the system and its component parts follows.

Integrated Surgical System

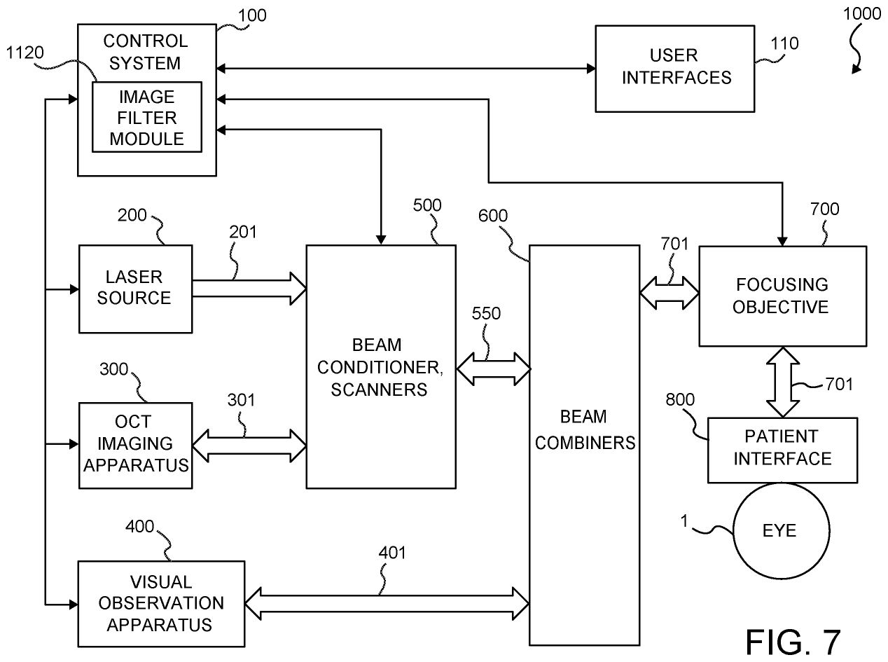

With reference to FIG. 7, an integrated surgical system 1000 for non-invasive glaucoma surgery includes a control system 100, a surgical component 200, a first imaging component 300 and an optional second imaging component 400. In the embodiment of FIG. 7, the surgical component 200 is a femtosecond laser source, the first imaging component 300 is an OCT imaging apparatus, and the optional second imaging component 400 is a visual observation apparatus, e.g., a microscope, for direct viewing or viewing with a camera. Other components of the integrated surgical system 1000 include beam conditioners and scanners 500, beam combiners 600, a focusing objective 700, and a patient interface 800.

The control system 100 may be a single computer or and plurality of interconnected computers configured to control the hardware and software components of the other components of the integrated surgical system 1000. A user interface 110 of the control system 100 accepts instructions from a user and displays information for observation by the user. Input information and commands from the user include but are not limited to system commands, motion controls for docking the patient's eye to the system, selection of pre-programmed or live generated surgical plans, navigating through menu choices, setting of surgical parameters, responses to system messages, determining and acceptance of surgical plans and commands to execute the surgical plan. Outputs from the system towards the user includes but are not limited to display of system parameters and messages, display of images of the eye, graphical, numerical and textual display of the surgical plan and the progress of the surgery.

The control system 100 is connected to the other components 200, 300, 400, 500 of the integrated surgical system 1000. Control signals from the control system 100 to the femtosecond laser source 200 function to control internal and external operation parameters of the laser source, including for example, power, repetition rate and beam shutter. Control signals from the control system 100 to the OCT imaging apparatus 300 function to control OCT beam scanning parameters, and the acquiring, analyzing and displaying of OCT images.

Laser beams 201 from the femtosecond laser source 200 and OCT beams 301 from the OCT imaging apparatus 300 are directed towards a unit of beam conditioners and scanners 500. Different kind of scanners can be used for the purpose of scanning the laser beam 201 and the OCT beam 301. For scanning transversal to a beam 201, 301, angular scanning galvanometer scanners are available for example from Cambridge Technology, Bedford, MA, Scanlab, Munich, Germany. To optimize scanning speed, the scanner mirrors are typically sized to the smallest size, which still support the required scanning angles and numerical apertures of the beams at the target locations. The ideal beam size at the scanners is typically different from the beam size of the laser beam 201 or the OCT beam 301, and different from what is needed at the entrance of a focusing objective 700. Therefore, beam conditioners are applied before, after or in between individual scanners. The beam conditioner and scanners 500 includes scanners for scanning the beam transversally and axially. Axial scanning changes the depth of the focus at the target region. Axial scanning can be performed by moving a lens axially in the beam path with a servo or stepper motor.

The laser beam 201 and the OCT beam 301 are combined with dichroic, polarization or other kind of beam combiners 600 to reach a common target volume or surgical volume in the eye. In an integrated surgical system 1000 having a femtosecond laser source 200, an OCT imaging apparatus 300, and a visual observation device 400, the individual beams 201, 301, 401 for each of these components may be individually optimized and may be collinear or non-collinear to one another. The beam combiner 600 uses dichroic or polarization beam splitters to split and recombine light with different wavelength and/or polarization. The beam combiner 600 may also include optics to change certain parameters of the individual beams 201, 301, 401 such as beam size, beam angle and divergence. Integrated visual illumination, observation or imaging devices assist the surgeon in docking the eye to the system and identifying surgical locations.

To resolve ocular tissue structures of the eye in sufficient detail, the imaging components 300, 400 of the integrated surgical system 1000 may provide an OCT beam and a visual observation beam having a spatial resolution of several micrometers. The resolution of the OCT beam is the spatial dimension of the smallest feature that can be recognized in the OCT image. It is determined mostly by the wavelength and the spectral bandwidth of the OCT source, the quality of the optics delivering the OCT beam to the target location in the eye, the numerical aperture of the OCT beam and the spatial resolution of the OCT imaging apparatus at the target location. In one embodiment, the OCT beam of the integrated surgical system has a resolution of no more than 5 μm.

Likewise, the surgical laser beam provided by the femtosecond laser source 200 may be delivered to targeted locations with several micrometer accuracy. The resolution of the laser beam is the spatial dimension of the smallest feature at the target location that can be modified by the laser beam without significantly affecting surrounding ocular tissue. It is determined mostly by the wavelength of the laser beam, the quality of the optics delivering the laser beam to target location in the eye, the numerical aperture of the laser beam, the energy of the laser pulses in the laser beam and the spatial resolution of the laser scanning system at the target location. In addition, to minimize the threshold energy of the laser for photo-disruptive interaction, the size of the laser spot should be no more than approximately 5 μm.

It should be noted that, while the visual observation beam 401 is acquired by the visual observation device 400 using fixed, non-scanning optics, the OCT beam 301 of the OCT imaging apparatus 300 is scanned laterally in two transversal directions. The laser beam 201 of the femtosecond laser source 200 is scanned in two lateral dimensions and the depth of the focus is scanned axially.

For practical embodiments, beam conditioning, scanning and combining the optical paths are certain functions performed on the laser, OCT and visual observation optical beams. Implementation of those functions may happen in a different order than what is indicated in FIG. 7. Specific optical hardware that manipulates the beams to implement those functions can have multiple arrangements with regards to how the optical hardware is arranged. They can be arranged in a way that they manipulate individual optical beams separately, in another embodiment one component may combine functions and manipulates different beams. For example, a single set of scanners can scan both the laser beam 201 and the OCT beam 301. In this case, separate beam conditioners set the beam parameters for the laser beam 201 and the OCT beam 301, then a beam combiner combines the two beams for a single set of scanners to scan the beams. While many combinations of optical hardware arrangements are possible for the integrated surgical system, the following section describes in detail an example arrangement.

Beam Delivery

In the following description, the term beam may—depending on the context—refer to one of a laser beam, an OCT beam, or a visual observation beam. A combined beam refers to two or more of a laser beam, an OCT beam, or a visual observation beam that are either collinearly combined or non-collinearly combined. Example combined beams include a combined OCT/laser beam, which is a collinear or non-colinear combination of an OCT beam and a laser beam, and a combined OCT/laser/visual beam, which is a collinear or non-collinear combination of an OCT beam, a laser beam, and a visual beam. In a collinearly combined beam, the different beams may be combined by dichroic or polarization beam splitters, and delivered along a same optical path through a multiplexed delivery of the different beams. In a non-collinear combined beam, the different beams are delivered at the same time along different optical paths that are separated spatially or by an angle between them. In the description to follow, any of the foregoing beams or combined beams may be generically referred to as a light beam. The terms distal and proximal may be used to designate the direction of travel of a beam, or the physical location of components relative to each other within the integrated surgical system. The distal direction refers to a direction toward the eye; thus an OCT beam output by the OCT imaging apparatus moves in the distal direction toward the eye. The proximal direction refers to a direction away from the eye; thus an OCT return beam from the eye moves in the proximal direction toward the OCT imaging apparatus.

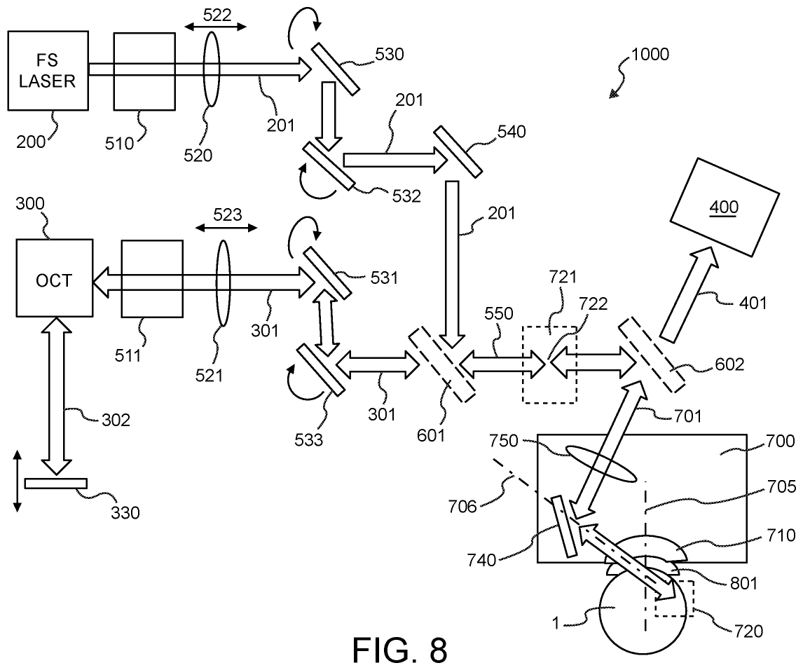

Referring to FIG. 8, an example integrated surgical system is configured to deliver each of a laser beam 201 and an OCT beam 301 in the distal direction toward an eye 1, and receive each of an OCT return beam and the visual observation beam 401 back from the eye 1. Regarding the delivery of a laser beam, a laser beam 201 output by the femtosecond laser source 200 passes through a beam conditioner 510 where the basic beam parameters, beam size, divergence are set. The beam conditioner 510 may also include additional functions, setting the beam power or pulse energy and shutter the beam to turn it on or off. After existing the beam conditioner 510, the laser beam 210 enters an axial scanning lens 520. The axial scanning lens 520, which may include a single lens or a group of lenses, is movable in the axial direction 522 by a servo motor, stepper motor or other control mechanism. Movement of the axial scanning lens 520 in the axial direction 522 changes the axial distance of the focus of the laser beam 210 at a focal point.

In accordance with a particular embodiment of the integrated surgical system, an intermediate focal point 722 is set to fall within, and is scannable in, the conjugate surgical volume 721, which is an image conjugate of the surgical volume 720, determined by the focusing objective 700. The surgical volume 720 is the spatial extent of the region of interest within the eye where imaging and surgery is performed. For glaucoma surgery, the surgical volume 720 is the vicinity of the irido-corneal angle 13 of the eye.

A pair of transverse scanning mirrors 530, 532 rotated by a galvanometer scanner scan the laser beam 201 in two essentially orthogonal transversal directions, e.g., in the x and y directions. Then the laser beam 201 is directed towards a dichroic or polarization beam splitter 540 where it is reflected toward a beam combining mirror 601 configured to combine the laser beam 201 with an OCT beam 301.

Regarding delivery of an OCT beam, an OCT beam 301 output by the OCT imaging apparatus 300 passes through a beam conditioner 511, an axially moveable focusing lens 521 and a transversal scanner with scanning mirrors 531 and 533. The focusing lens 521 is used set the focal position of the OCT beam in the conjugate surgical volume 721 and the real surgical volume 720. The focusing lens 521 is not scanned for obtaining an OCT axial scan. Axial spatial information of the OCT image is obtained by Fourier transforming the spectrum of the interferometrically recombined OCT return beam 301 and reference beams 302. However, the focusing lens 521 can be used to re-adjust the focus when the surgical volume 720 is divided into several axial segments. This way the optimal imaging spatial resolution of the OCT image can be extended beyond the Rayleigh range of the OCT signal beam, at the expense of time spent on scanning at multiple ranges.

Proceeding in the distal direction toward the eye 1, after the scanning mirrors 531 and 533, the OCT beam 301 is combined with the laser beam 201 by the beam combiner mirror 601. The OCT beam 301 and laser beam 201 components of the combined laser/OCT beam 550 are multiplexed and travel in the same direction to be focused at an intermediate focal point 722 within the conjugate surgical volume 721. After having been focused in the conjugate surgical volume 721, the combined laser/OCT beam 550 propagates to a second beam combining mirror 602 where it is combined with a visual observation beam 401 to form a combined laser/OCT/visual beam 701.

The combined laser/OCT/visual beam 701 traveling in the distal direction then passes through the focusing objective 700, and a window 801 of a patient interface, where the intermediate focal point 722 of the laser beam within the conjugate surgical volume 721 is re-imaged into a focal point in the surgical volume 720. The focusing objective 700 re-images the intermediate focal point 722, through the window 801 of a patient interface, into the ocular tissue within the surgical volume 720.

A scattered OCT return beam 301 from the ocular tissue travels in the proximal direction to return to the OCT imaging apparatus 300 along the same paths just described, in reverse order. The reference beam 302 of the OCT imaging apparatus 300, passes through a reference delay optical path and return to the OCT imaging apparatus from a moveable mirror 330. The reference beam 302 is combined interferometrically with the OCT return beam 301 on its return within the OCT imaging apparatus 300. The amount of delay in the reference delay optical path is adjustable by moving the moveable mirror 330 to equalize the optical paths of the OCT return beam 301 and the reference beam 302. For best axial OCT resolution, the OCT return beam 301 and the reference beam 302 are also dispersion compensated to equalize the group velocity dispersion within the two arms of the OCT interferometer.

When the combined laser/OCT/visual beam 701 is delivered through the cornea 3 and the anterior chamber 7, the combined beam passes through posterior and anterior surface of the cornea at a steep angle, far from normal incidence. These surfaces in the path of the combined laser/OCT/visual beam 701 create excessive astigmatism and coma aberrations that need to be compensated for.

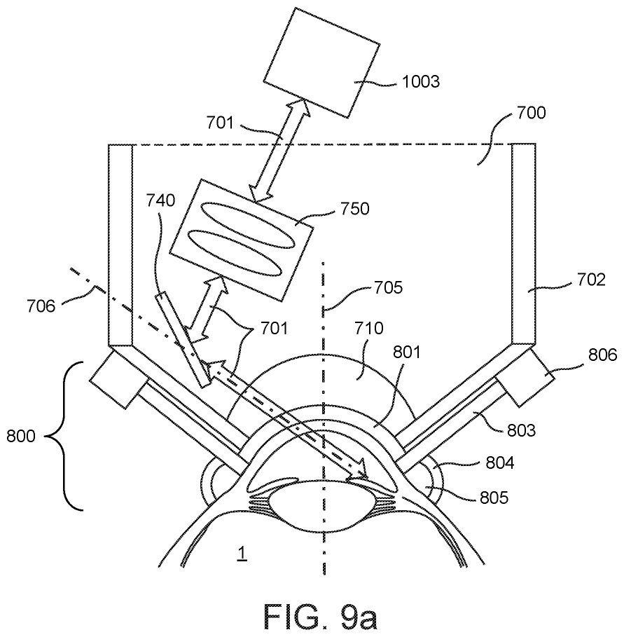

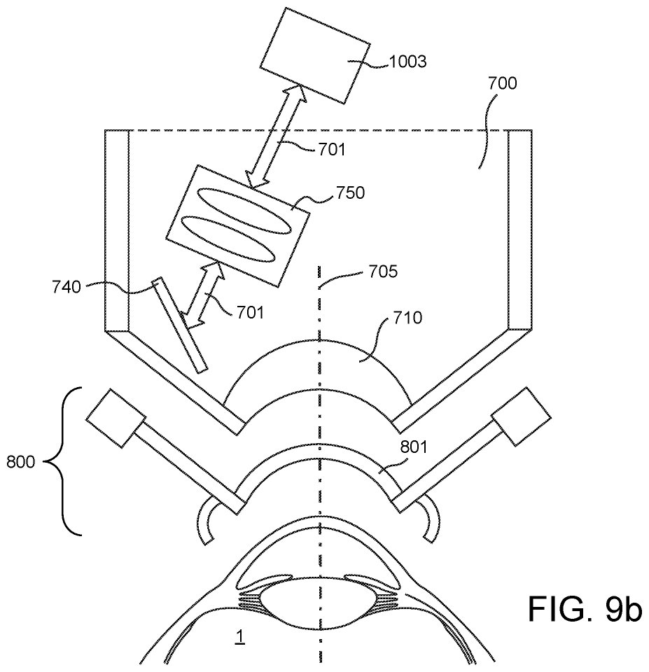

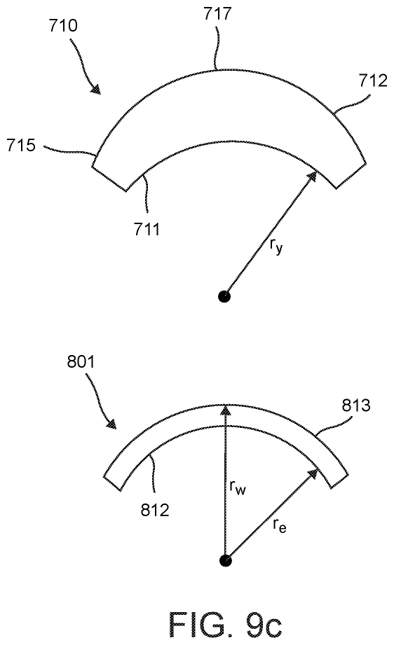

With reference to FIGS. 9a and 9b, in an embodiment of the integrated surgical system 1000, optical components of the focusing objective 700 and patient interface 800 are configured to minimize spatial and chromatic aberrations and spatial and chromatic distortions. FIG. 9a shows a configuration when both the eye 1, the patient interface 800 and the focusing objective 700 all coupled together. FIG. 9b shows a configuration when both the eye 1, the patient interface 800 and the focusing objective 700 all detached from one another.

The patient interface 800 optically and physically couples the eye 1 to the focusing objective 700, which in turn optically couples with other optic components of the integrated surgical system 1000. The patient interface 800 serves multiple functions. It immobilizes the eye relative to components of the integrated surgical system; creates a sterile barrier between the components and the patient; and provides optical access between the eye and the instrument. The patient interface 800 is a sterile, single use disposable device and it is coupled detachably to the eye 1 and to the focusing objective 700 of the integrated surgical system 1000.

The patient interface 800 includes a window 801 having an eye-facing, concave surface 812 and an objective-facing, convex surface 813 opposite the concave surface. The window 801 thus has a meniscus form. With reference to FIG. 9c, the concave surface 812 is characterized by a radius of curvature rₑ, while the convex surface 813 is characterized by a radius of curvature r_{w}. The concave surface 812 is configured to couple to the eye, either through a direct contact or through index matching material, liquid or gel, placed in between the concave surface 812 and the eye 1. The window 801 may be formed of glass and has a refractive index n_{w}. In one embodiment, the window 801 is formed of fused silica and has a refractive index n_{w} of 1.45. Fused silica has the lowest index from common inexpensive glasses. Fluoropolymers such as the Teflon AF are another class of low index materials that have refractive indices lower than fused silica, but their optical quality is inferior to glasses and they are relatively expensive for high volume production. In another embodiment the window 801 is formed of the common glass BK7 and has a refractive index n_{w} of 1.50. A radiation resistant version of this glass, BK7G18 from Schott AG, Mainz, Germany, allows gamma sterilization of the patient interface 800 without the gamma radiation altering the optical properties of the window 801.

Returning to FIGS. 9a and 9b, the window 801 is surrounded by a wall 803 of the patient interface 800 and an immobilization device, such as a suction ring 804. When the suction ring 804 is in contact with the eye 1, an annular cavity 805 is formed between the suction ring and the eye. When vacuum applied to the suction ring 804 and the cavity via a vacuum tube a vacuum pump (not shown in FIGS. 9a and 9b), vacuum forces between the eye and the suction ring attach the eye to the patient interface 800 during surgery. Removing the vacuum releases or detach the eye 1.

The end of the patient interface 800 opposite the eye 1 includes an attachment interface 806 configured to attach to the housing 702 of the focusing objective 700 to thereby affix the position of the eye relative to the other components of the integrated surgical system 1000. The attachment interface 806 can work with mechanical, vacuum, magnetic or other principles and it is also detachable from the integrated surgical system.

The focusing objective 700 includes an aspheric exit lens 710 having an eye-facing, concave surface 711 and a convex surface 712 opposite the concave surface. The exit lens 710 thus has a meniscus form. While the exit lens 710 shown in FIGS. 9a and 9b is an aspheric lens giving more design freedom, in other configurations the exit lens may be a spherical lens. Alternatively, constructing the exit lens 710 as a compound lens, as opposed to a singlet, allows more design freedom to optimize the optics while preserving the main characteristics of the optical system as presented here. With reference to FIG. 9c, the concave surface 711 is characterized by a radius of curvature r_{y}, while the convex surface 712 is characterized by an aspheric shape. The aspheric convex surface 712 in combination with the spherical concave surface 711 result in an exit lens 710 having varying thickness, with the outer perimeter edges 715 of the lens being thinner than the central, apex region 717 of the lens. The concave surface 711 is configured to couple to the convex surface 813 of the window 801. In one embodiment, the exit lens 710 is formed of fused silica and has a refractive index nₓ of 1.45.

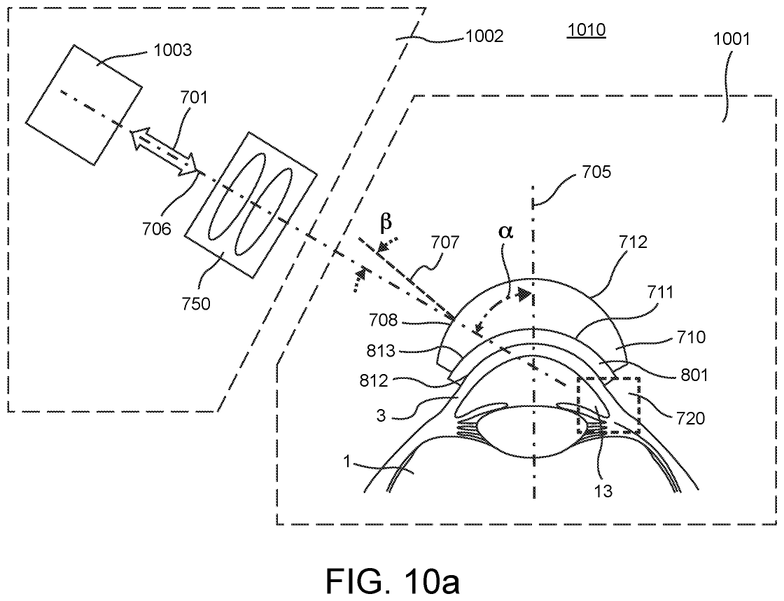

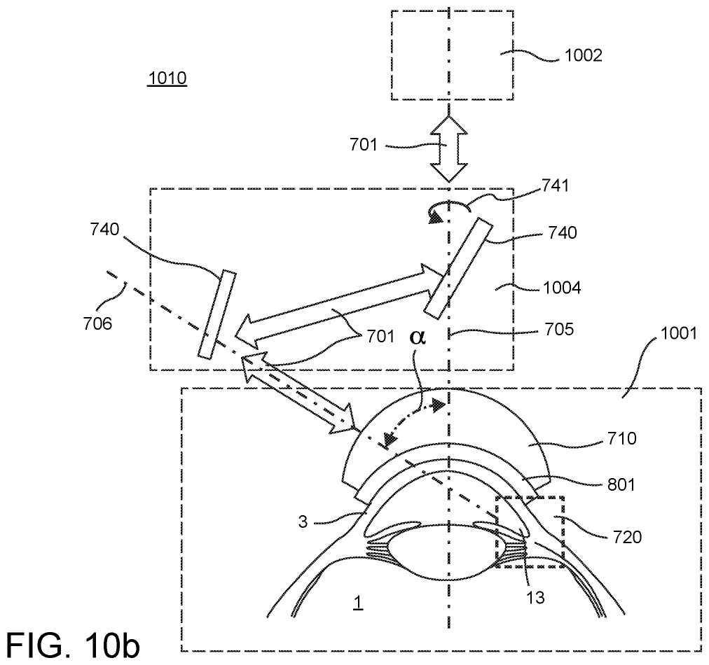

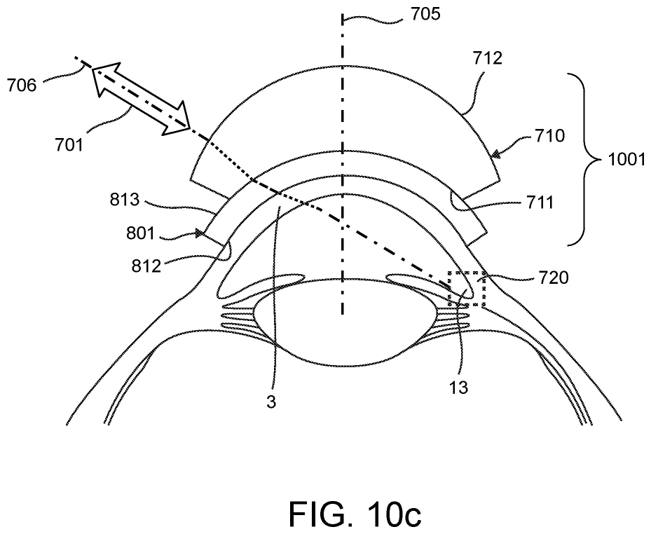

FIGS. 10a and 10b are schematic illustrations of components of the integrated surgical system of FIGS. 7 and 8 functionally arranged to form an optical system 1010 having a first optical subsystem 1001 and a second optical subsystem 1002 that enable access to a surgical volume 720 in the irido-corneal angle. Each of FIGS. 10a and 10b include components of the focusing objective 700 and the patient interface 800 of FIG. 9a. However, for simplicity, the entirety of the focusing objective and the patient interface are not included in FIGS. 10a and 10b. Also, for additional simplicity in FIG. 10a, the planar beam-folding mirror 740 of FIGS. 9a and 9b is not included and the combined laser/OCT/visual beam 701 shown in FIG. 9a is unfolded or straightened out. It is understood by those skilled in the art that adding or removing planar beam folding mirrors does not alter the principal working of the optical system formed by the first optical subsystem and the second optical subsystem. FIG. 10c is a schematic illustration of a beam passing through the first optical subsystem of FIGS. 10a and 10b.

With reference to FIG. 10a, a first optical subsystem 1001 of the integrated surgical system 1000 includes the exit lens 710 of a focusing objective 700 and the window 801 of a patient interface 800. The exit lens 710 and the window 801 are arranged relative to each other to define a first optical axis 705. The first optical subsystem 1001 is configured to receive a beam, e.g., a combined laser/OCT/visual beam 701, incident at the convex surface 712 of the exit lens 710 along a second optical axis 706, and to direct the beam toward a surgical volume 720 in the irido-corneal angle 13 of the eye.

During a surgical procedure, the first optical subsystem 1001 may be assembled by interfacing the convex surface 813 of the window 801 with the concave surface 711 of the exit lens 710. To this end, a focusing objective 700 is docked together with a patient interface 800. As a result, the concave surface 711 of the exit lens 710 is coupled to the convex surface 813 of the window 801. The coupling may be by direct contact or through a layer of index matching fluid. For example, when docking the patient interface 800 to focusing objective 700, a drop of index matching fluid can be applied between the contacting surfaces to eliminate any air gap that may be between the two surfaces 711, 813 to thereby help pass the combined laser/OCT/visual beam 701 through the gap with minimal Fresnel reflection and distortion.

In order to direct the beam toward the surgical volume 720 in the irido-corneal angle 13 of the eye, the first optical subsystem 1001 is designed to account for refraction of the beam 701 as it passes through the exit lens 710, the window 801 and the cornea 3. To this end, and with reference to FIG. 10c, the refractive index nₓ of the exit lens 710 and the refractive index n_{w} of the window 801 are selected in view of the refractive index n_{c} of the cornea 3 to cause appropriate beam bending through the first optical subsystem 1001 so that when the beam 701 exits the subsystem and passes through the cornea 3, the beam path is generally aligned to fall within the irido-corneal angle 13.

Continuing with reference to FIG. 10c and beginning with the interface between the window 801 and the cornea 3. Too steep of an angle of incidence at the interface where the combined laser/OCT/visual beam 701 exits the window 801 and enters the cornea 3, i.e., at the interface between the concave surface 812 of the window and the convex surface of the cornea 3, can create excessive refraction and distortion. To minimize refraction and distortion at this interface, in one embodiment of the first optical subsystem 1001, the refractive index of the window 801 is closely matched to the index of the cornea 3. For example, as describe above with reference to FIGS. 9a and 9b, the window 801 may have a refractive index lower than 1.42 to closely match the cornea 3, which has a refractive index of 1.36.

Excessive refraction and distortion at the interface where the combined laser/OCT/visual beam 701 exits the window 801 and enters the cornea 3 may be further compensated for by controlling the bending of the beam 701 as it passed through the exit lens 710 and the window 801. To this end, in one embodiment of the first optical subsystem 1001 the index of refraction n_{w} of the window 801 is larger than each of the index of refraction nₓ of the exit lens 710 and the index of refraction n_{c} of the cornea 3. As a result, at the interface where the combined laser/OCT/visual beam 701 exits the exit lens 710 and enters the window 801, i.e., interface between the concave surface 711 of the exit lens and the convex surface 813 of the window, the beam passes through a refractive index change from high to low that cause the beam to bend in a first direction. Then, at the interface where the combined laser/OCT/visual beam 701 exits the window 801 and enters the cornea 3, i.e., interface between the concave surface 812 of the exit lens and the convex surface of the cornea, the beam passes through a refractive index change from low to high that cause the beam to bend in a second direction opposite the first direction.

The shape of the window 801 is chosen to be a meniscus lens. As such, the incidence angle of light has similar values on both surfaces 812, 813 of the window 801. The overall effect is that at the convex surface 813 the light bends away from the surface normal and at the concave surface 812 the light bends towards the surface normal. The effect is like when light passes through a plan parallel plate. Refraction on one surface of the plate is compensated by refraction on the other surface a light passing through the plate does not change its direction. Refraction at the entering, convex surface 712 of the exit lens 710 distal to the eye is minimized by setting the curvature of the entering surface such that angle of incidence β of light 701 at the entering surface is close to a surface normal 707 to the entering surface at the intersection point 708.

Here, the exit lens 710, the window 801, and the eye 1 are arranged as an axially symmetric system with a first optical axis 705. In practice, axial symmetry is an approximation because of manufacturing and alignment inaccuracies of the optical components, the natural deviation from symmetry of the eye and the inaccuracy of the alignment of the eye relative to the window 801 and the exit lens 710 in a clinical setting. But, for design and practical purposes the eye 1, the window 801, and the exit lens 710 are considered as an axially symmetric first optical subsystem 1001.

With continued reference to FIG. 10a, a second optical subsystem 1002 is optically coupled to the first optical subsystem 1001 at an angle α relative to the first optical axis 705 of the first optical subsystem 1001. The advantage of this arrangement is that the both optical subsystems 1001, 1002 can be designed at a much lower numerical aperture compared to a system where all optical components are designed on axis with a common optical axis.

The second optical subsystem 1002 includes a relay lens 750 that, as previously described with reference to FIG. 8, generates a conjugate surgical volume 721 of the surgical volume 720 within the eye. The second optical subsystem 1002 includes various other components collectively indicated as an optical subsystem step 1003. Referring to FIG. 8, these components may include a femtosecond laser source 200, an OCT imaging apparatus 300, a visual observation device 400, beam conditioners and scanners 500, and beam combiners 600.

The second optical subsystem 1002 may include mechanical parts (not shown) configured to rotate the entire subsystem around the first optical axis 705 of the first optical subsystem 1001. This allows optical access to the whole 360-degree circumference of the irido-corneal angle 13 of the eye 1.

With reference to FIG. 10b, flexibility in arranging the first and second optical subsystems 1001, 1002, relative to each other may be provided by an optical assembly 1004 interposed between the optical output of the second optical subsystem 1002 and the optical input of the first optical subsystem 1001. In one embodiment, the optical assembly 1004 may include one or more planar beam-folding mirrors 740, prisms (not shown) or optical gratings (not shown) configured to receive the optical output, e.g., combined laser/OCT/visual beam 701, of the second optical subsystem 1002, change or adjust the direction of the combined laser/OCT/visual beam, and direct the beam to the optical input of the first optical subsystem 1001 while preserving the angle α between the first optical axis 705 and the second optical axis 706.

In another configuration, the optical assembly 1004 of planar beam-folding mirrors 740 further includes mechanical parts (not shown) configured to rotate the assembly around the first optical axis 705 of the first optical subsystem 1001 while keeping the second optical subsystem 1002 stationary. Accordingly, the second optical axis 706 of the second optical subsystem 1002 can be rotated around the first optical axis 705 of the first optical subsystem 1001. This allows optical access to the whole 360-degree circumference of the irido-corneal angle 13 of the eye 1.

With considerations described above with reference to FIGS. 9a, 9b and 9c, the design of the first optical subsystem 1001 is optimized for angled optical access at an angle a relative to the first optical axis 705 of the first optical subsystem 1001. Optical access at the angle α compensates for optical aberrations of the first optical subsystem 1001. Table 1 shows the result of the optimization at access angle α=72 degrees with Zemax optical design software package. This design is a practical embodiment for image guided femtosecond glaucoma surgery.

TABLE 1

| | Structure and | Refractive | Radius | Center |
|---|---|---|---|---|
| Surface | Material | index | [mm] | Thickness [mm] |
| concave | Exit lens 710 of | 1.45 | −10 | 4.5 |
| surface | focusing objective. | | | |
| 711, | | | | |
| convex | Fused silica | | | |
| surface | | | | |
| 712 | | | | |
| concave | Window 801 of | 1.50 | −10.9 | 1.0 |
| surface | patient interface. | | | |
| 812, | | | | |
| convex | BK7G18 | | | |
| surface | | | | |
| 813 | | | | |
| 3 | Cornea | 1.36 | −7.83 | 0.54 |
| 8 | Aqueous humor | 1.32 | −6.53 | 3.5 |
| Target | Ophthalmic tissue | 1.38 | N/A | 0 to 1 mm |

This design produces diffraction limited focusing of 1030 nm wavelength laser beams and 850 nm wavelength OCT beams with numerical aperture (NA) up to 0.2. In one design, the optical aberrations of the first optical subsystem are compensated to a degree that the Strehl ratio of the first optical subsystem for a beam with numerical aperture larger than 0.15 at the irido-corneal angle is larger than 0.9. In another design, the optical aberrations of the first optical subsystem are partially compensated, the remaining uncompensated aberrations of the first optical system are compensated by the second optical subsystem to a degree that the Strehl ratio of the combined first and second optical subsystem for a beam with numerical aperture larger than 0.15 at the irido-corneal angle is larger than 0.9.

Laser Surgical Treatments

In accordance with embodiments disclosed herein, the integrated surgical system 1000 enables laser treatment of tissues, especially ocular tissue structures in the irido-corneal angle of the eye. In one embodiment, the laser is a femtosecond laser. A femtosecond laser provides highly localized, non-thermal photo-disruptive laser-tissue interaction with minimal collateral damage to surrounding ocular tissue. Photo-disruptive interaction of the laser is utilized in optically transparent tissue. The principal mechanism of laser energy deposition into the ocular tissue is not by absorption but by a highly nonlinear multiphoton process. This process is effective only at the focus of the pulsed laser where the peak intensity is high. Regions where the beam is traversed but not at the focus are not affected by the laser. Therefore, the interaction region with the ocular tissue is highly localized both transversally and axially along the laser beam.

Prior to laser treatment, the user specifies the size and shape of the treatment pattern to be scanned by the femtosecond laser beam. The specification of the treatment pattern includes the length, width, and depth of the pattern, as well as other parameters including: 1) the separation distance between adjacent 2D planes or layers of the treatment pattern, and 2) the separation between photo-disruption sites in a plane. The separation distance, also referred to as the layer separation, may be the same between each layer, or it may vary. Likewise, the separation between photo-disruption sites within a layer, also referred to as the spot distance, may be the same in each layer, or it may vary. For example, it is possible to vary the density of spots in a treatment pattern by specifying the spot separation discreetly for each layer or as function of the depth of the layer. Additional treatment pattern parameters may specify a setting for the level of laser energy to be applied at each photo-disruption site in a layer. An energy setting may be specified for each layer in the treatment pattern. For example, different layers may be produced using different surgical energy settings, or the same energy setting. The energy setting of each layer can be specified discreetly or as a continuous function of depth of the layer. In cases where the energy setting is the same across all layers in a treatment pattern, the layers have substantially the same thickness. In cases where the energy setting is the different among one or more layers in a treatment pattern, the one or more layers may have different thicknesses.

The specifications are inputted to a processor that analyzes the specification data to compute the 3-dimensional (3D) location of each photo-disruption site within the treatment pattern. In addition, for each 3D location the processor determines whether the delivery of optical energy to that location is sufficient to affect ocular tissue. As a result of this analysis, each photo-disruption site is assigned a four-dimensional coordinate. The first three coordinates define the 3D location of a single photo-disruption site. These first three coordinates may be represented in Cartesian, Spherical, or other coordinate representation. The fourth coordinate is a binary bit that determines whether the delivery of optical energy to the location specified by the proceeding three coordinates is sufficient to affect ocular tissue. The processor then saves the four-dimensional coordinate in a memory of the processor. The processor continues to compute and save a four-dimensional coordinate for each photo-disruption site within the treatment pattern until the pattern has been exhausted. The final result is N four-dimensional coordinates, where N is the total number of photo-disruption sites in the treatment pattern, saved into memory. The aggregate of these coordinates represents the computational specification of the treatment pattern broken down by individual sites within a treatment plane at a specified depth and by the separation between adjacent treatment planes.

During a laser surgical treatment, a laser focus is scanned in accordance with a computed specification of the treatment pattern saved in memory. For each photo-disruption site, the processor moves the scanning device to focus the femtosecond laser to the computed 3D location. The processor then checks the fourth coordinate to determine if the delivery of optical energy to the location should be sufficient to affect ocular tissue. If the fourth bit allows that such energy should be delivered, then processor enables the delivery of optical energy to the location sufficient to affect ocular tissue, and then reads the next set of coordinates for the treatment pattern. If the fourth bit prohibits such energy, then the processor implements additional components for preventing optical energy from affecting tissue at this location. In this manner, a number of photo-disruptions sites, or spots, affecting tissue in layers or planes, each plane being located at a different depth in ocular tissue is generated.

At instances during the scanning specified by the computed treatment pattern, the laser delivers optical energy sufficient to affect a cell or spot of ocular tissue. The size of a spot or cell is determined by the extent of the influence of laser-tissue interaction, and in the case of a femtosecond laser is on the order of a few micrometers (m). The cumulation of these spots over the course of scanning through a number of tissue planes results in a three-dimensional volume of ocular tissue comprising multiple sheets or layers of affected tissue.

Because the laser interaction volume is small, the interaction of ocular tissue with each laser shot of a repetitive laser breaks down ocular tissue locally at the focus of the laser. The pulse duration of the laser for photo-disruptive interaction in ocular tissue can range from several femtoseconds to several nanoseconds and pulse energies from several nanojoules to tens of microjoules. The laser pulses at the focus, through multiphoton processes, breaks down chemical bonds in the molecules, locally photo-dissociate tissue material and create gas bubbles in wet tissue. The breakdown of tissue material and mechanical stress from bubble formation fragments the tissue and create clean continuous cuts when the laser pulses are laid down in proximity to one another along geometrical lines and surfaces.

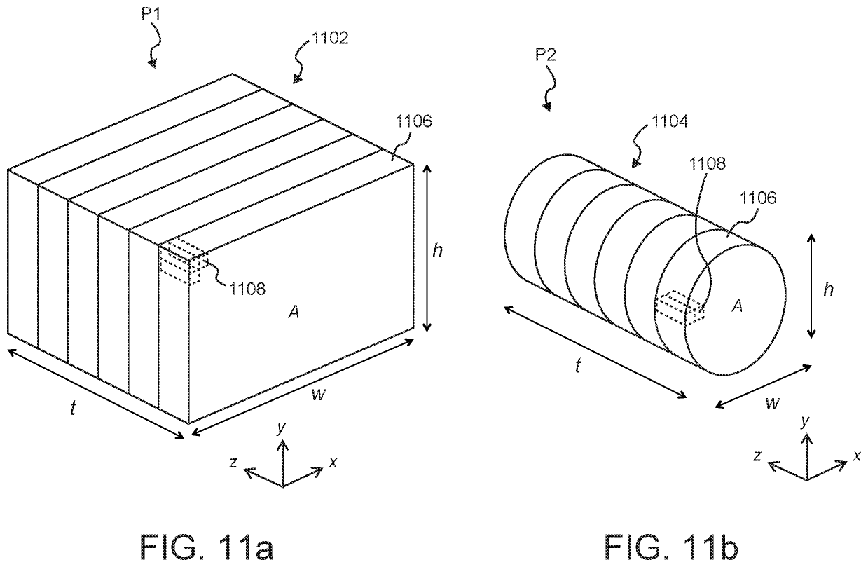

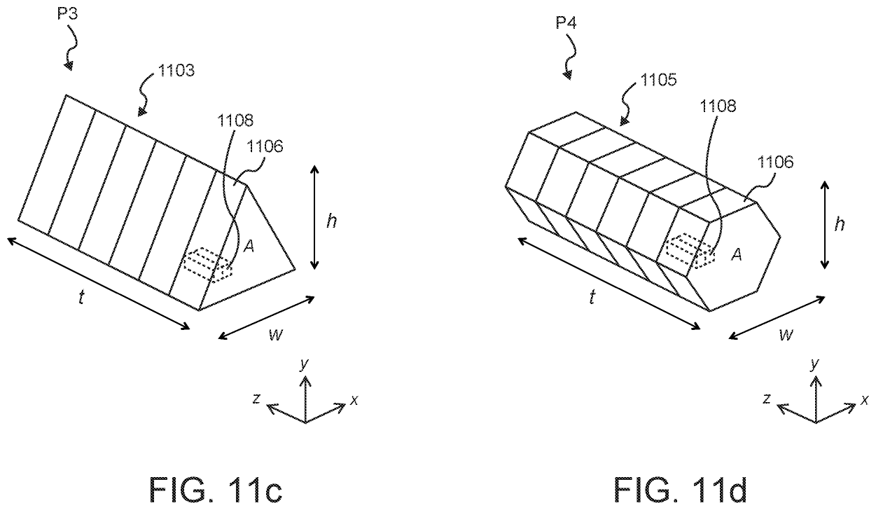

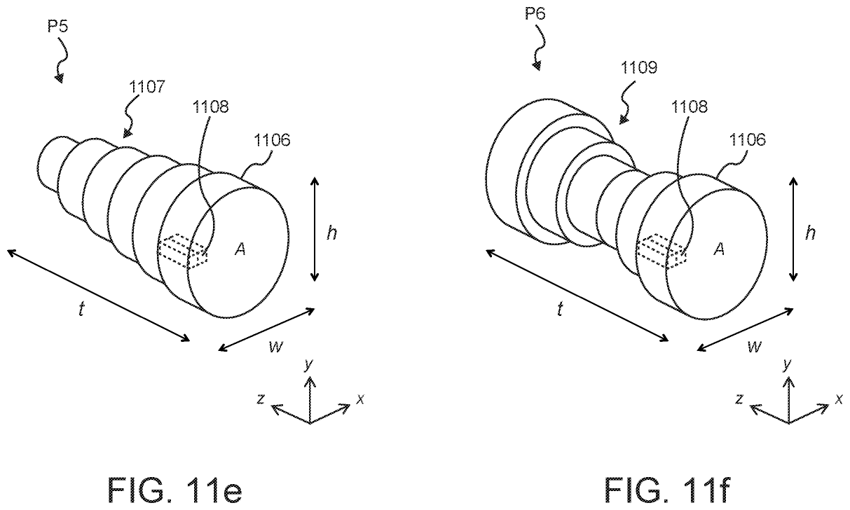

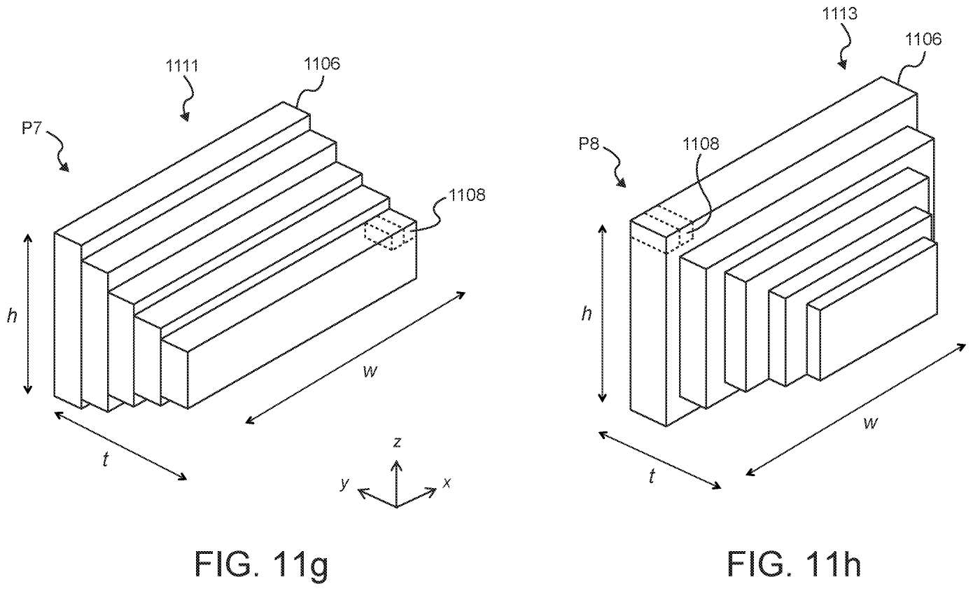

The instances at which optical energy is delivered during a laser treatment are defined by a laser treatment pattern. With reference to FIGS. 11a through 11h, a treatment pattern P1, P2, P3, P4, P5, P6, P7, and P8 may be in the form of any geometric structure, e.g., an extended rectangle 1102, a cylinder 1104, an extended triangle 1103, and extended hexagon 1105, a cone 1107, a hourglass 1109, a trapezoidal prism 1111, and a trapezoidal pyramid 1113, etc., and may encompass a number of sheets or layers, referred to herein as treatment planes 1106, adjacent each other. Each treatment plane 1106, in turn, encompasses individual cells 1108 arranged in regularly spaced rows and columns. A treatment pattern P1, P2, P3, P4, P5, P6, P7, P8 may be characterized by x, y, z dimensions, with x, y, z coordinates of the cells 1108 being calculated sequentially from neighbor to neighbor in the order of a column location (x coordinate), a row location (y coordinate), and a layer location (z coordinate). A treatment pattern P1, P2, P3, P4, P5, P6, P7, P8 as such, defines a three-dimensional model of ocular tissue to be modified by a laser. In addition to providing a treatment for glaucoma, treatment patterns P1, P2, P3, P4, P5, P6, P7, P8 can be shaped to maximize the clinical benefit of such treatments. For example a three-dimensional trapezoidal prism 1111 or a three-dimensional trapezoidal pyramid 1113 have the added benefit of treating a relatively large area of Schlemm's canal 18 while creating a relatively small aperture through the trabecular meshwork 12 into the anterior chamber 7.

The treatment patterns P1, P2, P3, P4, P5, P6, P7 shown in FIGS. 11a through 11g have layers of equal thickness, while the treatment pattern P8 shown in FIG. 11h has layers of varying thicknesses. As describe above, layer thickness may be determined or controlled by parameters that specify a setting for the level of laser energy to be applied at each photo-disruption site in a layer, with a higher energy level producing a thicker layer.

The integrated surgical system 1000 disclosed herein is configured to scan its laser focus in a scanning pattern. The scanning pattern may be characterized by a geometry and movement of the laser focus. For example, the geometry may be symmetric like a rectangle, square or circle or it may be non-symmetric and may be defined by x and y dimensions. Movement of the laser focus through a geometry may be raster like, circular, spiral, zig-zag, etc. Accordingly, the scanning pattern of the integrated surgical system 1000 may be referred to as having a raster pattern, a circular pattern, a spiral pattern, or a non-symmetric pattern.

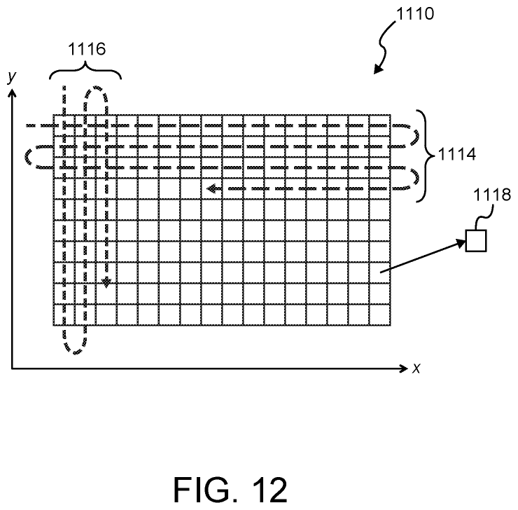

With reference to FIG. 12, scanning pattern 1110 having a rectangular geometry and a raster movement pattern is shown. The geometry of the scanning pattern 1110 is defined by a horizontal dimension x and a vertical dimension y, which dimensions are typically pre-set or programmed in the control system 100 of the integrated surgical system 1000 for a given laser. For example, a femtosecond laser may have a scanning pattern 1110, where x=800 μm and y=300 μm. During laser treatment, the laser focus is located at a depth in tissue and then scanned either in a horizontal pattern 1114 or a vertical pattern 1116 to define a layer or plane of laser scanning.

The integrated surgical system 1000 is also configured to deliver optical energy during laser scanning. To this end, the system is programmed to deliver a pulse shot of the laser at each of a plurality of instances 1118 as pre-computed by a processor during a horizontal scan 1114 or vertical scan 1116, whichever the case may be. For example, with reference to FIG. 12, during a horizontal scan 1114 a laser shot sufficient to affect a cell or spot of ocular tissue may be delivered to tissue at each instance 1118 represented by a square. As described above, these instances 1118 are pre-computed by the processor. The delivery of laser shots at each instance 1118 is repeated for each row in a horizontal scan 1114. When scanning is complete, the multiple instances 1118 of optical energy delivery produce a layer of laser-affected tissue. The thickness of the layer of laser-affected tissue is a function of the energy of the laser shots. For example, a femtosecond laser with a pulse energy between 3-5 μJ. may result in a layer thickness of between 3-4 μm.

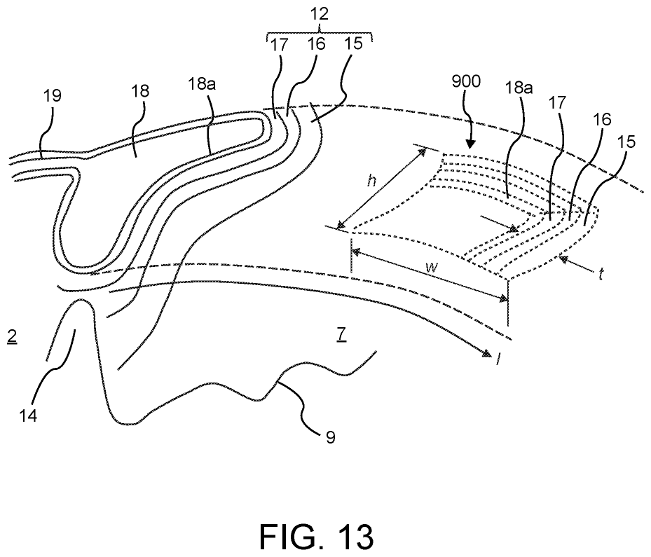

With reference to FIGS. 13-20, a treatment pattern P1, P2, P3, P4, P5, P6, P7, P8 is typically defined by a set of surgical parameters. The surgical parameters may include a treatment area A that represents a surface area or layer of ocular tissue through which the laser will travel. The treatment area A is determined by the treatment height, h, and the width or lateral extent of the treatment, w. The width or lateral extent may be defined in terms of a measure or length around the circumferential angle. For example, the width w may be defined in terms of an angle, e.g., 90 degrees, around the circumferential angle.

The surgical parameters may also include a treatment thickness t that represents the level to which the laser will cut into the ocular tissue from a distal extent 60 or border of the treatment volume at or near Schlemm's canal 18 to a proximal extent 62 or border at or near the surface of the trabecular meshwork 12. Thus, a laser applied in accordance with a treatment pattern may affect or produce a surgical volume that resembles the three-dimensional model of the treatment pattern.

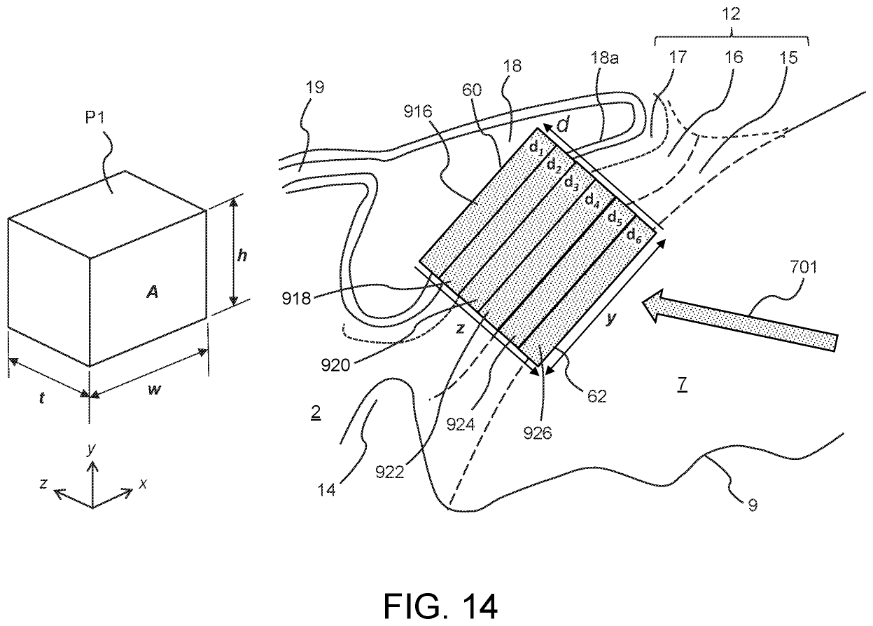

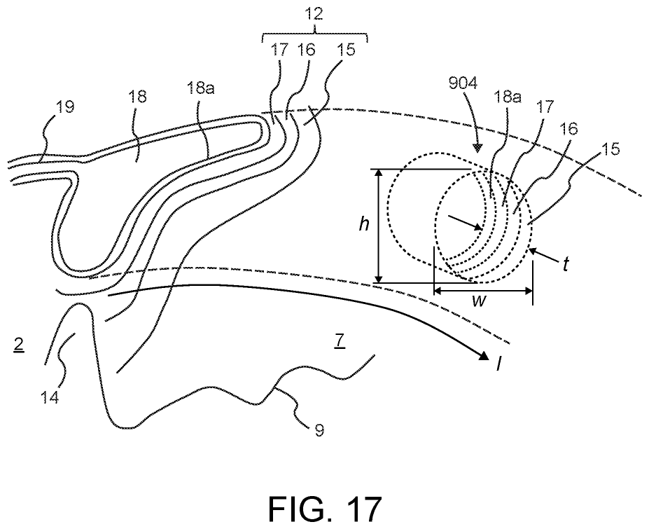

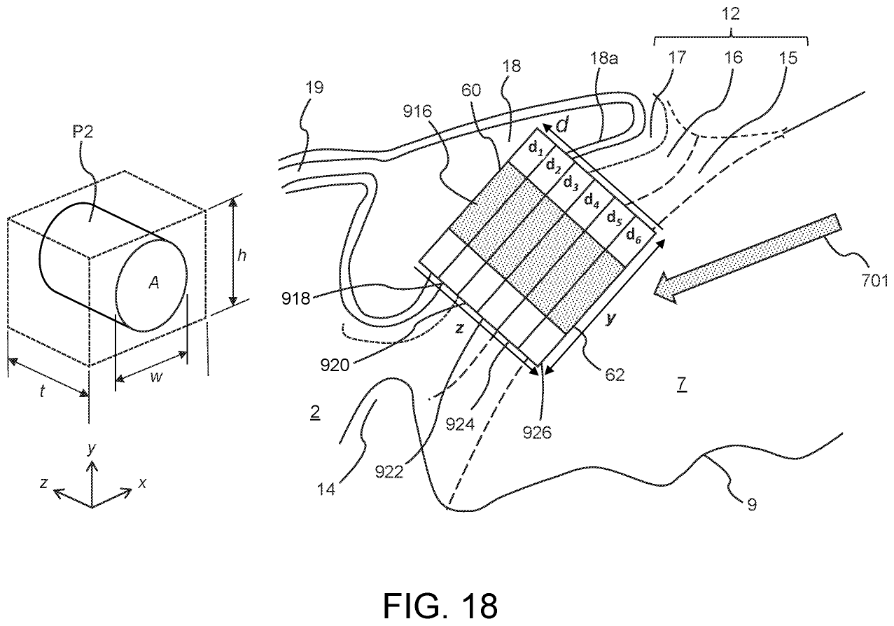

Additional surgical parameters define the placement of the surgical volume or affected volume within the eye. For example, placement parameters may include one or more of a location l, as shown in FIGS. 13 and 17, that represents where the treatment is to occur relative to the circumferential angle of the eye, and a treatment depth d, as shown in FIGS. 14 and 18, that represents a position of the three-dimensional model of ocular tissue relative to a reference eye structure. In the following, the treatment depth d is shown and described relative to the region where the anterior chamber 7 meets the trabecular meshwork 12. Together, the treatment pattern and the placement parameters define a treatment plan.

Different shapes and sizes of volumes of ocular tissue may be affected by controlling the delivery of optical energy to tissue during laser scanning based on different corresponding treatment patterns. For example, with reference to FIGS. 13-16, a cubic surgical volume 900 of ocular tissue may be treated in accordance with a treatment pattern P1, like the one shown in FIG. 11a. The surgical volume 900 of ocular tissue may comprise portions of the trabecular meshwork 12 and the Schlemm's canal 18. For example, the surgical volume 900 of ocular tissue shown in FIG. 13 includes portions of the uveal 15, the corneoscleral meshwork 16, the juxtacanalicular tissue 17, and the inner wall 18a of the Schlemm's canal 18.

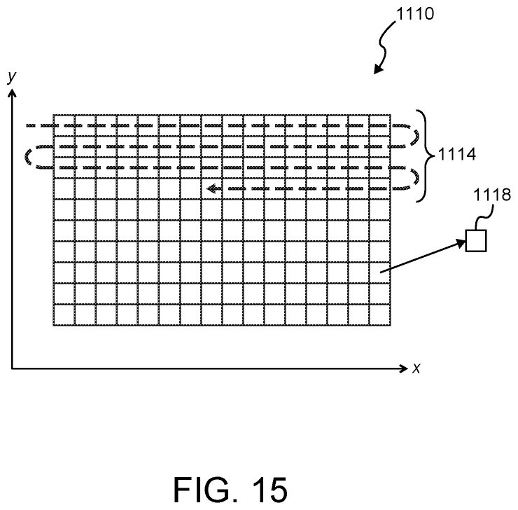

With reference to FIG. 14, during a laser scanning procedure in accordance with treatment pattern P1, the focus of the laser beam 701 is initially located at a depth d₁. This depth d₁ places the laser focus in an initial layer 916 of tissue. Once the laser focus is positioned at the initial depth d₁, the focus is scanned in accordance with a horizontal scan 1114 through the scanning pattern 1110, with optical energy being delivered at each instance 1118 during the scan, as shown in FIG. 15. These instances 1118 where optical energy is delivered to tissue result in the photodisruption of the initial layer 916 of tissue and define an initial treatment plane at the initial layer of tissue.

With continued reference to FIG. 14, the focus of the laser beam 701 is then moved in the z direction toward the anterior chamber 7 to a subsequent depth d₂. The distance between the initial depth d₁ and the subsequent depth d₂ is referred to the layer separation distance. The subsequent depth d₂ places the laser focus at a subsequent layer 918 of tissue. Once the laser focus is positioned at the subsequent depth d₂, the focus is scanned in accordance with a horizontal scan 1114 through the scanning pattern 1110, with optical energy being delivered at each instance 1118 during the scan, as shown in FIG. 15. These instances 1118 of optical energy delivery result in the photodisruption of the subsequent layer 918 of tissue and define a subsequent treatment plane at the subsequent layer of tissue. The foregoing movement of the focus of the laser beam 701 and laser scanning and optical energy delivery is repeated at depths d₃, d₄, d₅, and d₆ resulting in photodisruption of the subsequent layers 918, 920, 922, 924, 926 of tissue.

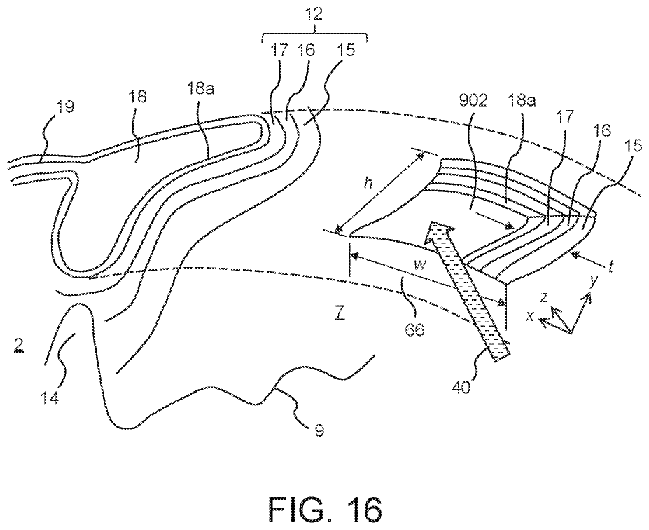

With reference to FIG. 16, photodisruption of the multiple layers forms an opening 902 between the anterior chamber 7 and the Schlemm's canal 18, thus completing the laser treatment procedure. The opening 902 resulting from laser application of the treatment pattern P1 resembles the surgical volume 900 and is characterized by an area A and thickness t similar to those of the surgical volume and the treatment pattern. The thickness t of the resulting opening 902 extends from the anterior chamber 7 and through the inner wall 18a of the Schlemm's canal 18, while the area A defines the cross-section size of the opening 902.

With reference to FIGS. 17-20, a cylindrical surgical volume 904 of ocular tissue may be treated in accordance with a treatment pattern P2, like the one shown in FIG. 11b. The surgical volume 904 of ocular tissue may comprise portions of the trabecular meshwork 12 and the Schlemm's canal 18. For example, the surgical volume 900 of ocular tissue shown in FIG. 17 includes portions of the uveal 15, the corneoscleral meshwork 16, the juxtacanalicular tissue 17, and the inner wall 18a of the Schlemm's canal 18.

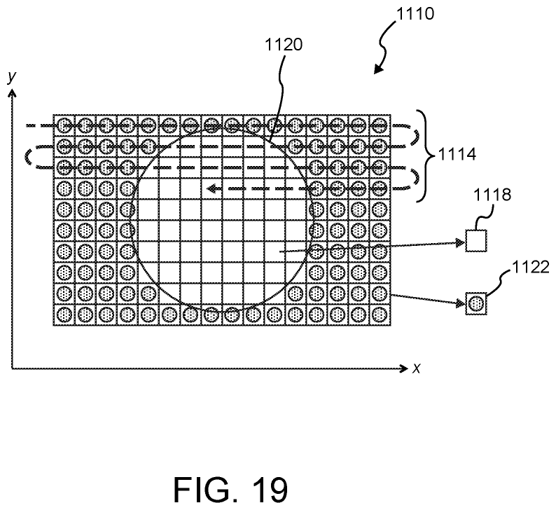

With reference to FIG. 18, during a laser scanning procedure in accordance with treatment pattern P2, the focus of the laser beam 701 is initially located at a depth d₁. This depth d₁ places the laser focus in an initial layer 916 of tissue. Once the laser focus is positioned at the initial depth d₁, the focus is scanned in accordance with a horizontal scan 1114 through the scanning pattern 1110. During the scan 1114 an image filter is applied to control the delivery of optical energy. The image filter may be selected from a library of image filters included in the image filter module 1120 of the control system 100. Based on information included in an image filter, the control system 100 functions to prevent optical energy from affecting tissue during certain instances 1122 pre-computed by the processor during the scan, while allowing for the delivery of optical energy at other instances 1118 pre-computed by the processor, as shown for example in FIG. 19. The instances 1118 where optical energy is delivered to tissue result in the photodisruption of the initial layer 916 of tissue and define an initial treatment plane at the initial layer of tissue.

To prevent optical energy from affecting tissue, the control system 100 may be configured to control a femtosecond laser source 200 to cause the laser source to not output a laser pulse during these instances 1122. In other words, the control system 100 may blank select output pulses from the laser source 200 that coincide with these instances 1122 during the scanning of the laser through the scanning pattern 1110. Alternatively, the control system 100 may cause the femtosecond laser source 200 to reduce the energy of its outputs pulses so that the pulses output during these instances 1122 deliver optical energy that is insufficient to affect tissue.

With continued reference to FIG. 18, the focus of the laser beam 701 is then moved in the z direction toward the anterior chamber 7 to a subsequent depth d₂. The subsequent depth d₂ places the laser focus at a subsequent layer 918 of tissue. Once the laser focus is positioned at the subsequent depth d₂, the focus is scanned in accordance with a horizontal scan 1114 through the scanning pattern 1110, with optical energy being delivered to tissue at select instances 1118 during the scan and being prevented from affecting tissue at other instances 1122 during the scan. The instances 1118 of optical energy delivery to tissue result in the photodisruption of the subsequent layer 918 of tissue and define a subsequent treatment plane at the subsequent layer of tissue. The foregoing movement of the focus of the laser beam 701 and laser scanning and optical energy delivery and prevention is repeated at depths d₃, d₄, d₅, and d₆ resulting in photodisruption of the subsequent layers 918, 920, 922, 924, 926 of tissue.

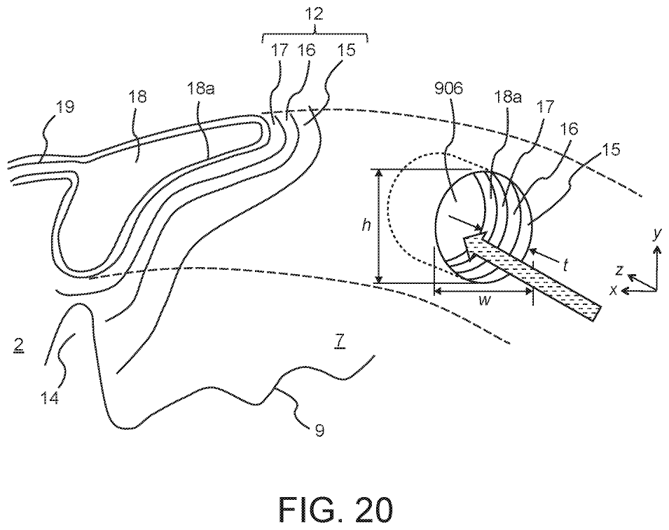

With reference to FIG. 20, photodisruption of the multiple layers forms an opening 906 between the anterior chamber 7 and the Schlemm's canal 18, thus completing the laser treatment procedure. The opening 902 resulting from laser application of the treatment pattern P1 resembles the surgical volume 904 and is characterized by an area A and thickness t similar to those of the surgical volume and the treatment pattern. The thickness t of the resulting opening 906 extends from the anterior chamber 7 and through the inner wall 18a of the Schlemm's canal 18, while the area A defines the cross-section size of the opening 902.

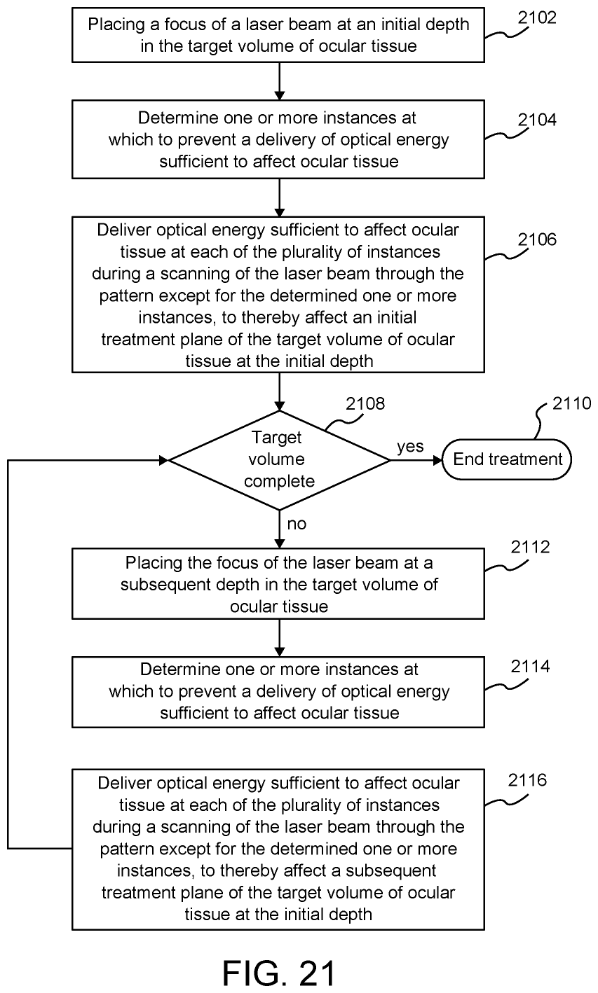

FIG. 21 is a flow chart of a method of treating a target volume of ocular tissue of an irido-corneal angle of an eye with a laser. The method may be performed by the integrated surgical system 1000 of FIG. 7. The integrated surgical system 1000 includes a laser source 200 configured to output a laser beam 201 and to deliver optical energy sufficient to affect ocular tissue at each of a plurality of instances pre-computed by the processor during a scanning of the laser beam through a scanning pattern 1110.

At block 2102, and with additional reference to FIGS. 17-20, the integrated surgical system 1000 controls light from a laser source 200 so that the focus of a laser beam 201/701 is at an initial depth d₁ in a target volume of ocular tissue 904.

At block 2104, the integrated surgical system 1000 determines one or more instances at which to prevent a delivery of optical energy sufficient to affect ocular tissue. For example, the control system 200 of the integrated surgical system 1000 may obtain information from an image filter module 1120 that identifies the one or more instances 1122 during a scanning of the laser beam 701 at which the delivery of optical energy sufficient to affect ocular tissue is to be prevented. The information may be embodied in an image filter that is pre-computed by a processor and that represents a treatment pattern for the volume of ocular tissue 904 to be treated, such as the treatment patterns shown in FIGS. 11a-11h.

A library of image filters may be stored in the image filter module 1120. Visual representations of the treatment patterns defined by the image filters may be made available for viewing and selection through a user interface 110 of the integrated surgical system 1000. Upon selection of an image filter, the control system 100 reads a file corresponding to the selected image filter to extract the information that identifies the one or more instances pre-computed by the processor 1122 during a scanning of the laser at which the delivery of optical energy sufficient to affect ocular tissue is to be prevented.

In one embodiment, the image filter corresponds to a 3D CAD model file. The 3D CAD model file may be, for example, in the form of a stereo lithography (STL) file, corresponding to 3D layer information. These 3D CAD model files or STL files may be derived from a variety of software applications that include, but are not limited to Alibre, Geomagic Design, AutoCAD, CadKey, KeyCreator, I-DEAS, Inventor, IronCAD, Mechanical Desktop, ProE, Rhino, SolidDesigner, SolidEdge, SolidWorks, Think3, and Unigraphics. The 3D layer information comprises multiple layers or planes of 2D information that collectively define a 3D CAD model. For example, the 3D CAD model, may correspond to one of the 3D treatment patterns shown in FIGS. 11a-11f. This 3D layer information is used by the control system 100 to determine which laser pulses to blank during scanning of the laser beam 701 through a scanning pattern 1110, where each layer provides 2D information, e.g., x, y coordinates, specifying the location or instances 1122 where a laser pulse is blanked. Through the blanking of laser pulses during a scanning of the laser beam 701, only specific points or cells of tissue in a 2D layer of tissue are affected to thereby define a treatment plane. Collectively, treatment of tissue in accordance with multiple layers of 2D information included in the 3D CAD model form a 3D volume of treated tissue corresponding to the 3D CAD model.

In another embodiment, the image filter corresponds to one or more OCT images. OCT images can contain 2D and/or 3D information that can be converted through an algorithm, such as edge detection, blob detection, etc., that is converted to an on/off binary file. This binary file is used by the control system 100 to determine at which instances 1122 laser pulses are off and at which instances laser pulses are on during the scanning of the laser beam 701 through a scanning pattern 1110.

In another embodiment, the image filter corresponds to an Excel spreadsheet that stores on/off binary information in a matrix of spreadsheet cells, where each cell maps to an x, y coordinate, or instance 1118 of a scanning pattern 1110. This binary file is used by the control system 100 to determine at which instances 1122 laser pulses are off and at which instances laser pulses are on during the scanning of the laser beam through the scanning pattern.

In another embodiment, the image filter corresponds to an electronic tabulation of spatial coordinates. A software/mathematical algorithm can produce x, y coordinates along with pulse on/off information that can be tabulated in a format that can be used by the control system 100 to determine at which instances 1122 laser pulses are off and at which instances laser pulses are on during the scanning of the laser beam 701 through a scanning pattern 1110. In another embodiment, the image filter corresponds to an image in a standard image format such as BMP, JPG, PNG, TIFF, etc. These images may be digitized by the control system 100 into a 2D array of cells, where each cell maps to an x, y coordinate, or instance 1118 of a scanning pattern 1110. A cell in the digitized image that contains a portion of the image may be designated an on pulse, while a cell in the digitized image that is empty or blank, i.e., does not contain a portion of the image, may be designated an off pulse.

Continuing with FIG. 21, at block 2106, the integrated surgical system 1000 delivers optical energy sufficient to affect ocular tissue at each of the plurality of instances 1118 during a scanning of the laser beam through the scanning pattern 1110—except for the determined one or more instances 1122. Scanning and delivering optical energy in this manner affects an initial treatment plane 916 of the target volume of ocular tissue 904 at the initial depth d₁. To prevent optical energy from affecting tissue, the control system 100 may be configured to control a femtosecond laser source 200 to cause the laser source to not output a laser pulse during these instances 1122. In other words, the control system 100 may blank select output pulses from the laser source 200 that coincide with these instances 1122 during the scanning of the laser through the scanning pattern 1110.

In another configuration, the integrated surgical system 1000 may include additional components for preventing optical energy from affecting tissue. For example, a fast, mechanical shutter may be located at the output of the laser source 200 and the control system 100 may be configured to open and close the shutter in order to allow or block the passage of laser beam 201 from the laser source 200. In another embodiment, an acousto-optic modulator or a combination of polarizers and Pockels cells may be located at the output of the laser source 200 and controlled by the control system 100 to allow or block the passage of laser beam 201. In yet another embodiment, the focusing objective 700 may include an optical element that focuses and de-focuses laser beam 701 and the control system 100 may be configured to control the optical element in a manner that allows or blocks the passage of laser beam 201. Alternatively, the control system 100 may cause the laser source 200 to reduce the energy of laser pulses output during these instances 1122 to a level that is insufficient to affect tissue.

Returning to FIG. 21, at block 2108, the integrated surgical system 1000 determines if treatment of the target volume of ocular tissue 904 is complete. If treatment is complete, the integrated surgical system 1000 ends treatment at block 2110. If treatment is not complete, the integrated surgical system 1000 continues treatment by affecting at least one subsequent treatment plane 918, 920, 922, 924, 926 of the volume of ocular tissue 904. To this end, at block 2112, the integrated surgical system 1000 places the focus of the laser beam 701 at a subsequent depth d₂, d₃, d₄, d₅, d₆ in the target volume of ocular tissue 904.

At block 2114, the integrated surgical system 1000 determines one or more instances 1122 at which to prevent a delivery of optical energy sufficient to affect ocular tissue. For example, the control system 200 of the integrated surgical system 1000 may obtain information from an image filter module 1120 that identifies the one or more instances 1122 during a scanning of the laser beam 701 at which the delivery of optical energy sufficient to affect ocular tissue is to be prevented. The information may be embodied in an image filter that is pre-computed by a processor and that represents a treatment pattern for the volume of ocular tissue 904 to be treated, such as the treatment patterns shown in FIGS. 11a-11h.

At block 2116, the integrated surgical system 1000 delivers optical energy sufficient to affect ocular tissue at each of the plurality of instances 1118 during a scanning of the laser beam through the scanning pattern 1110—except for the determined one or more instances 1122. Scanning and delivering optical energy in this manner affects a subsequent treatment plane 918, 920, 922, 924, 926 of the target volume of ocular tissue 904 at the subsequent depth d₂, d₃, d₄, d₅, d₆. The initial treatment plane 916 and the one or more subsequent treatment planes 918, 920, 922, 924, 926 are adjacent relative to each other, and the combination of the initial treatment plane and the one or more subsequent treatment planes define the target volume of ocular tissue 904.

The determined instances 1122 where delivery of optical energy that affects tissue is prevented while scanning the laser beam 701 through the scanning pattern 1110 at the initial depth d₁ and at the one or more subsequent depths d₂, d₃, d₄, d₅, d₆ may be identical. In such cases, the image filter defining the instances 1122 may be the same for each treatment plane. For example, with reference to FIG. 11b the image filter may be a circle of the same size for each treatment plane, in which case the resulting treated volume of ocular tissue 904 is a cylinder.

The determined instances 1122 where delivery of optical energy that affects tissue is prevented while scanning the laser beam 701 through the scanning pattern 1110 at the initial depth d₁ and at the one or more subsequent depths d₂, d₃, d₄, d₅, d₆ may not be identical. In such cases, the image filter defining the instances 1122 may be different for each treatment plane. For example, with reference to FIG. 11e, the image filter may be a circle for teach treatment plane, where the diameter of the circle is successively smaller. In this case, the resulting treated volume of ocular tissue 904 is a cone.

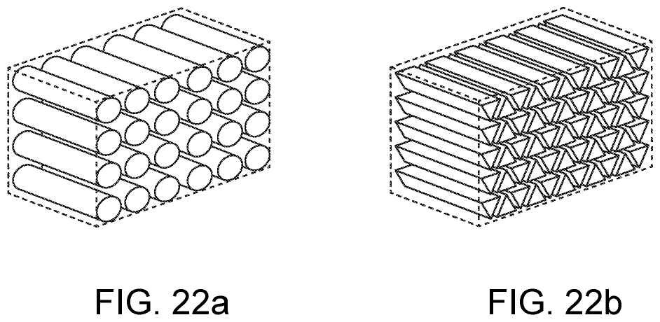

While the laser treatment patterns P1, P2, P3, P4, P5, P6, P7, P8 shown in FIGS. 11a-11h are in the form of a single geometric structure, a treatment pattern may comprise a number of individual geometric structure. For example, with reference to FIG. 22a, a laser treatment pattern may include a number of cylinders positioned adjacent each other in a 4×6 array. With reference to FIG. 22b, a laser treatment pattern may include a number of extended triangles positioned adjacent each other in a 4×6 array, where adjacent triangles are inverted relative to each other.

3D laser treatment patterns, such as those shown in FIGS. 11a-11h, 22a, and 22b may be pre-computed by a processor based on a specified three-dimensional (3D) scanning pattern of a laser surgical system and stored in memory as image files. The pre-computed 3D treatment patterns include a plurality of instances where optical energy sufficient to affect ocular tissue is delivered, and other instances where such optical energy is not delivered. Once generated, a treatment pattern may be used by an apparatus to control the delivery of optical energy during a scanning of a laser beam 701 through the 3D scanning pattern.

With reference to FIG. 7, such an apparatus may be embodied by the control system 100 and the image file module 1120 of the control system. The image file module 1120 may be a memory that stores a number of different image files, e.g., 3D CAD software files, a stereo lithography files, image files, spreadsheet files, and electronic tabulations of spatial coordinates, corresponding to different 3D treatment patterns. The control system 100 includes a controller coupled to the memory. The controller is configured to obtain an image file corresponding to a pre-computed 3D treatment pattern from the memory. Information in the image file of the 3D treatment pattern identifies first instances of the 3D scanning pattern at which a delivery of optical energy sufficient to affect ocular tissue is to be allowed, and second instances of the 3D scanning pattern at which a delivery optical energy sufficient to affect ocular tissue is to be prevented. Based on the information in the image file, the controller delivers optical energy sufficient to affect ocular tissue at each of the first instances during a scanning of the laser beam through the 3D scanning pattern, and prevents a delivery of optical energy sufficient to affect ocular tissue at each of the second instances during a scanning of the laser beam through the 3D scanning pattern.

The information in the image file may include 3D coordinate locations of the first instances and the second instances within the 3D scanning pattern. This information may further include a binary bit of a first value for each of the first instances, and a binary bit of a second value for each of the second instances. The information may also include an optical energy level parameter for each of the first instances. The optical energy level parameter may be the same for each of the first instances, or it may be different for at least one of the first instances. The 3D treatment pattern may be defined by a plurality of treatment layers, and the information may include a layer separation parameter that specifies the distance between a first treatment layer of the plurality of treatment layers and a second treatment layer of the plurality of treatment layers, which may be adjacent the first treatment layer.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure, but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A method of treating a target volume of ocular tissue of an irido-corneal angle of an eye with a laser source configured to deliver optical energy sufficient to affect ocular tissue at each of a plurality of instances during a scanning of a laser beam through a scanning pattern, the method comprising:
placing a focus of the laser beam at an initial depth in the target volume of ocular tissue;
determining one or more instances at which to prevent a delivery of optical energy sufficient to affect ocular tissue; and
delivering optical energy sufficient to affect ocular tissue at each of the plurality of instances during a scanning of the laser beam through the scanning pattern except for the determined one or more instances, to thereby affect an initial treatment plane of the target volume of ocular tissue at the initial depth.

2. The method of claim 1, wherein determining one or more of the instances comprises obtaining information that identifies the one or more instances.

3. The method of claim 2, wherein the information is embodied in one of a 3D CAD software file, a stereo lithography file, an image file, a plurality of image files, an spreadsheet file, and an electronic tabulation of spatial coordinates.

4. The method of claim 1, wherein the scanning pattern through which the laser beam is scanned comprises one of a raster pattern, a circular pattern, a spiral pattern, or a non-symmetric pattern.

5. The method of claim 1, further comprising:
placing the focus of the laser beam at a subsequent depth in the target volume of ocular tissue;
determining one or more instances at which to prevent a delivery of optical energy sufficient to affect ocular tissue; and
delivering optical energy sufficient to affect ocular tissue at each of the plurality of instances during a scanning of the laser beam through the scanning pattern except for the determined one or more instances, to thereby affect a subsequent treatment plane of the target volume of ocular tissue at the subsequent depth.

6. The method of claim 5, wherein the initial treatment plane and the subsequent treatment plane are adjacent each other.

7. The method of claim 5, wherein a combination of the initial treatment plane and the subsequent treatment plane defines the target volume of ocular tissue.

8. The method of claim 5, wherein the determined while scanning the laser beam through the scanning pattern at the initial depth and the determined instances while scanning the laser beam through the scanning pattern at the subsequent depth are identical.

9. The method of claim 5, wherein the determined instances while scanning the laser beam through the scanning pattern at the initial depth and the determined instances while scanning the laser beam through the scanning pattern at the subsequent depth are not identical.

10. An integrated surgical system for treating a target volume of ocular tissue of an irido-corneal angle of an eye, the system comprising:
a first optical subsystem including a focusing objective configured to be coupled to a cornea of the eye;
a second optical subsystem including: a laser source configured to output a laser beam and to deliver optical energy sufficient to affect ocular tissue at each of a plurality of instances during a scanning of the laser beam through a scanning pattern, and a plurality of components configured to one or more of condition, scan, and direct the laser beam; and
a control system coupled to one or more of the first optical subsystem and the second optical subsystem and configured to control the focus, scan and optical energy delivery of the laser source to:
place a focus of the beam laser at an initial depth in the target volume of ocular tissue,
determine one or more instances at which to prevent a delivery of optical energy sufficient to affect ocular tissue; and
deliver optical energy sufficient to affect ocular tissue at each of the plurality of instances during a scanning of the laser beam through the scanning pattern except for the determined one or more instances, to thereby affect an initial treatment plane of the target volume of ocular tissue at the initial depth.

11. The system of claim 10, wherein the control system determines one or more of the instances by being further configured to obtain information that identifies the one or more instances.

12. The system of claim 11, wherein the information is embodied in one of a 3D CAD software file, a stereo lithography file, an image file, a spreadsheet file, and an electronic tabulation of spatial coordinates.

13. The system of claim 10, wherein the scanning pattern through which the laser beam is scanned comprises one of a raster pattern, a circular pattern, a spiral pattern, or a non-symmetric pattern.

14. The system of claim 10, wherein the control system is further configured to:
place the focus of the laser beam at a subsequent depth in the target volume of ocular tissue;
determine one or more instances at which to prevent a delivery of optical energy sufficient to affect ocular tissue; and
deliver optical energy sufficient to affect ocular tissue at each of the plurality of instances during a scanning of the laser beam through the scanning pattern except for the determined one or more instances, to thereby affect a subsequent treatment plane of the target volume of ocular tissue at the subsequent depth.

15. The system of claim 14, wherein the initial treatment plane and the subsequent treatment plane are adjacent each other.

16. The system of claim 14, wherein a combination of the initial treatment plane and the subsequent treatment plane defines the target volume of ocular tissue.

17. The system of claim 14, wherein the determined instances while scanning the laser beam through the scanning pattern at the initial depth and the determined instances while scanning the laser beam through the scanning pattern at the subsequent depth are identical.

18. The system of claim 14, wherein the determined instances while scanning the laser beam through the scanning pattern at the initial depth and the determined instances while scanning the laser beam through the scanning pattern at the subsequent depth are not identical.

19. An apparatus for controlling delivery of optical energy during a scanning of a laser beam through a three-dimensional (3D) scanning pattern, the apparatus comprising:
a memory; and
a controller coupled to the memory and configured to:
obtain information corresponding to a pre-computed 3D treatment pattern that identifies first instances of the 3D scanning pattern at which a delivery of optical energy sufficient to affect ocular tissue is to be allowed, and second instances of the 3D scanning pattern at which a delivery optical energy sufficient to affect ocular tissue is to be prevented;
deliver optical energy sufficient to affect ocular tissue at each of the first instances during a scanning of the laser beam through the 3D scanning pattern; and
prevent a delivery of optical energy sufficient to affect ocular tissue at each of the second instances during a scanning of the laser beam through the 3D scanning pattern.

20. The apparatus of claim 19, wherein the information comprises 3D coordinate locations of the first instances and the second instances within the 3D scanning pattern.

21. The apparatus of claim 20, wherein the information further comprises a binary bit of a first value for each of the first instances, and a binary bit of a second value for each of the second instances.

22. The apparatus of claim 19, wherein the information further comprises an optical energy level parameter for each of the first instances.

23. The apparatus of claim 22, wherein the optical energy level parameter is the same for each of the first instances.

24. The apparatus of claim 22, wherein the optical energy level parameter is different for at least one of the first instances.

25. The apparatus of claim 19, wherein the 3D treatment pattern comprises a plurality of treatment layers, and the information comprises a layer separation parameter that specifies a distance between a first treatment layer of the plurality of treatment layers and a second treatment layer of the plurality of treatment layers.
